SEQUENCE LISTING (86 sequences)

SEQ ID NO: 1 - protein, 17 aa, Homo sapiens
CATSDRGQGANWDEQFF

SEQ ID NO: 2 - protein, 13 aa, Homo sapiens
CASTLGGGTEAFF

SEQ ID NO: 3 - protein, 15 aa, Homo sapiens
CSARDLLAETYEQYF

SEQ ID NO: 4 - protein, 13 aa, Homo sapiens
CASSSSDTDTQYF

SEQ ID NO: 5 - protein, 18 aa, Homo sapiens
CSVEGSLGRALRANEQFF

SEQ ID NO: 6 - protein, 11 aa, Homo sapiens
CATHGGEKLFF

SEQ ID NO: 7 - protein, 14 aa, Homo sapiens
CASSYVGLGSPLHF

SEQ ID NO: 8 - protein, 11 aa, Homo sapiens
CSGQANTEAFF

SEQ ID NO: 9 - protein, 17 aa, Homo sapiens
CASSPTTGLKTRSGYTF

SEQ ID NO: 10 - protein, 12 aa, Homo sapiens
CSEGSPYNEQFF

SEQ ID NO: 11 - protein, 14 aa, Homo sapiens
CASSNGFHFNTLYF

SEQ ID NO: 12 - protein, 13 aa, Homo sapiens
CASSLGGGDTQYF

SEQ ID NO: 13 - protein, 13 aa, Homo sapiens
CASSFAGTDTQYF

SEQ ID NO: 14 - protein, 15 aa, Homo sapiens
CASSLGEGSPGELFF

SEQ ID NO: 15 - protein, 15 aa, Homo sapiens
CASSQEPNWNTEAFF

SEQ ID NO: 16 - protein, 14 aa, Homo sapiens
CASSFQGPGYGYTF

SEQ ID NO: 17 - protein, 14 aa, Homo sapiens
CSARDTTWGLEQYF

SEQ ID NO: 18 - protein, 14 aa, Homo sapiens
CATKPSGSTDTQYF

SEQ ID NO: 19 - protein, 14 aa, Homo sapiens
CSARDEGIGYEQYF

SEQ ID NO: 20 - protein, 12 aa, Homo sapiens
CASSSGPGELFF

SEQ ID NO: 21 - protein, 20 aa, Homo sapiens
CARRTLVIVRRFYSGNTIYF

SEQ ID NO: 22 - protein, 16 aa, Homo sapiens
CSARDLIGSQTYEQYF

SEQ ID NO: 23 - protein, 16 aa, Homo sapiens
CSARDPIGTESYEQYF

SEQ ID NO: 24 - protein, 14 aa, Homo sapiens
CSARDRAGRSPLHF

SEQ ID NO: 25 - protein, 14 aa, Homo sapiens
CSVEESSGIYEQYF

SEQ ID NO: 26 - protein, 15 aa, Homo sapiens
CSAREDGGQTYEQYF

SEQ ID NO: 27 - protein, 13 aa, Homo sapiens
CASSWAGPVEQYF

SEQ ID NO: 28 - protein, 13 aa, Homo sapiens
CASSSQGRAEQYF

SEQ ID NO: 29 - protein, 14 aa, Homo sapiens
CASSSRDSLYEQYF

SEQ ID NO: 30 - protein, 15 aa, Homo sapiens
CASSLGIISGQPQHF

SEQ ID NO: 31 - protein, 13 aa, Homo sapiens
CASSNTGGYTQYF

SEQ ID NO: 32 - protein, 15 aa, Homo sapiens
CASSQGLLLDNEQFF

SEQ ID NO: 33 - protein, 17 aa, Homo sapiens
CASSSPMDSGDTDTQYF

SEQ ID NO: 34 - protein, 13 aa, Homo sapiens
CASSPRSGVPQHF

SEQ ID NO: 35 - protein, 17 aa, Homo sapiens
CASSFVREEGSTDTQYF

SEQ ID NO: 36 - protein, 13 aa, Homo sapiens
CSARGTESYEQYF

SEQ ID NO: 37 - protein, 15 aa, Homo sapiens
CASWPGEGFGETQYF

SEQ ID NO: 38 - protein, 13 aa, Homo sapiens
CSGWGQGDEKLFF

SEQ ID NO: 39 - protein, 15 aa, Homo sapiens
CASSEYTSGNQPQHF

SEQ ID NO: 40 - protein, 16 aa, Homo sapiens
CSARDLWTGETYEQYF

SEQ ID NO: 41 - protein, 14 aa, Homo sapiens
CSATGLAGLGEQFF

SEQ ID NO: 42 - protein, 15 aa, Homo sapiens
CATSDLGTGVGEQFF

SEQ ID NO: 43 - protein, 13 aa, Homo sapiens
CSVGPGSTGELFF

SEQ ID NO: 44 - protein, 12 aa, Homo sapiens
CASSPTGEKLFF

SEQ ID NO: 45 - protein, 16 aa, Homo sapiens
CASSQEGGTWGDGYTF

SEQ ID NO: 46 - protein, 20 aa, Homo sapiens
CATSDLLLAGGRSSYNEQFF

SEQ ID NO: 47 - protein, 14 aa, Homo sapiens
CASSEAASGRPQTF

SEQ ID NO: 48 - protein, 19 aa, Homo sapiens
CATSDATAGTSGSLYEQYF

SEQ ID NO: 49 - protein, 14 aa, Homo sapiens
CASSLTGLGQPQHF

SEQ ID NO: 50 - protein, 14 aa, Homo sapiens
CASSPAVLSYEQYF

SEQ ID NO: 51 - protein, 15 aa, Homo sapiens
CSARESLAETYEQYF

SEQ ID NO: 52 - protein, 14 aa, Homo sapiens
CASSPGLTANVLTF

SEQ ID NO: 53 - protein, 14 aa, Homo sapiens
CASSLGLAGNEQYF

SEQ ID NO: 54 - protein, 14 aa, Homo sapiens
CASSNGFHFNTQYF

SEQ ID NO: 55 - protein, 14 aa, Homo sapiens
CASSLGILTDTQYF

SEQ ID NO: 56 - protein, 13 aa, Homo sapiens
CASSFQPVDTQYF

SEQ ID NO: 57 - protein, 13 aa, Homo sapiens
CSASEGIGQPQHF

SEQ ID NO: 58 - protein, 12 aa, Homo sapiens
CASSVSGGEQFF

SEQ ID NO: 59 - protein, 17 aa, Homo sapiens
CATSDRGQGANWDEQFF

SEQ ID NO: 60 - protein, 13 aa, Homo sapiens
CASTLGGGTEAFF

SEQ ID NO: 61 - protein, 15 aa, Homo sapiens
CSARDLLAETYEQYF

SEQ ID NO: 62 - protein, 13 aa, Homo sapiens
CASSSSDTDTQYF

SEQ ID NO: 63 - protein, 18 aa, Homo sapiens
CSVEGSLGRALRANEQFF

SEQ ID NO: 64 - protein, 11 aa, Homo sapiens
CATHGGEKLFF

SEQ ID NO: 65 - protein, 14 aa, Homo sapiens
CASSYVGLGSPLHF

SEQ ID NO: 66 - protein, 11 aa, Homo sapiens
CSGQANTEAFF

SEQ ID NO: 67 - protein, 17 aa, Homo sapiens
CASSPTTGLKTRSGYTF

SEQ ID NO: 68 - protein, 12 aa, Homo sapiens
CSEGSPYNEQFF

SEQ ID NO: 69 - protein, 14 aa, Homo sapiens
CASSNGFHFNTLYF

SEQ ID NO: 70 - protein, 13 aa, Homo sapiens
CASSLGGGDTQYF

SEQ ID NO: 71 - protein, 10 aa, Homo sapiens
EAAGIGILTV

SEQ ID NO: 72 - protein, 10 aa, Homo sapiens
LLLGIGILVL

SEQ ID NO: 73 - protein, 10 aa, Homo sapiens
NLSALGIFST

SEQ ID NO: 74 - protein, 9 aa, Homo sapiens
RLVDDFLLV

SEQ ID NO: 75 - protein, 9 aa, Homo sapiens
ALKDVEERV

SEQ ID NO: 76 - protein, 10 aa, Homo sapiens
ITSAIGVLPV

SEQ ID NO: 77 - protein, 10 aa, Homo sapiens
ITSAIGILPV

SEQ ID NO: 78 - protein, 10 aa, Homo sapiens
MTSAIGVLPV

SEQ ID NO: 79 - protein, 10 aa, Homo sapiens
QTSAIGVLPV

SEQ ID NO: 80 - protein, 10 aa, Homo sapiens
MTSAIGILPV

SEQ ID NO: 81 - protein, 10 aa, Homo sapiens
LTSAIGVLPV

SEQ ID NO: 82 - protein, 10 aa, Homo sapiens
ITSGIGVLPV

SEQ ID NO: 83 - protein, 10 aa, Homo sapiens
ITSAIGVLPI

SEQ ID NO: 84 - protein, 10 aa, Homo sapiens
QTSAIGILPV

SEQ ID NO: 85 - protein, 10 aa, Homo sapiens
ITSAIGVLFV

SEQ ID NO: 86 - protein, 10 aa, Homo sapiens
ALWGPDPAAA

(19) United States
(12) Patent Application Publication
(10) Pub. No.: US 2022/0233662 A1
(43) Pub. Date: Jul. 28, 2022

(54) NOVEL PEPTIDE

(71) Applicant: University College Cardiff Consultants Ltd., Cardiff South Wales (GB)

(72) Inventors: Andrew SEWELL, Cardiff South Glamorgan (GB); Garry DOLTON, Cardiff South Glamorgan (GB)

(21) Appl. No.: 17/646,562

(22) Filed: Dec. 30, 2021

Related U.S. Application Data
(60) Provisional application No. 63/132,158, filed on Dec. 30, 2020.

(51) Int. Cl. A61K 39/00; A61P 35/00

(52) U.S. Cl. CPC A61K 39/0011; A61K 2039/5158; A61K 2039/5154; A61P 35/00

(57) ABSTRACT

The present disclosure relates to a new peptide; a new fusion polypeptide, a polynucleotide or vector encoding same; a pharmaceutical composition or immunogenic composition or vaccine comprising said peptide; use of said peptide, vector, pharmaceutical composition, immunogenic composition or vaccine to treat cancer; a method of treating cancer using said peptide, fusion polypeptide, polynucleotide, vector, pharmaceutical composition, immunogenic composition or vaccine; an ex vivo method of stimulating and/or amplifying T-cells; and a combination therapeutic for the treatment of cancer comprising said peptide fusion polypeptide, polynucleotide, vector, pharmaceutical composition, immunogenic composition or vaccine.

### CROSS-REFERENCE

This application claims the benefit of U.S. Provisional Application No. 63/132,158, filed Dec. 30, 2020; the entire contents of which is hereby incorporated by reference.

### SEQUENCE LISTING

The sequence listing attached herewith named, “EBW-002US (188321)-Sequence Listing_ST25.txt” (16,384 bytes) and created on Dec. 30, 2020, is herein incorporated by reference in its entirety.

### TECHNICAL FIELD

The present disclosure relates inter alia to a new peptide; a new fusion polypeptide; a polynucleotide or vector encoding the same; a pharmaceutical composition or immunogenic composition or vaccine comprising said peptide or fusion polypeptide; use of said peptide, fusion polypeptide, polypeptide, vector, pharmaceutical composition, immunogenic composition or vaccine to treat cancer; a method of treating cancer using said peptide, fusion polypeptide, polynucleotide, vector, pharmaceutical composition, immunogenic composition or vaccine; an ex vivo method of stimulating and/or amplifying T-cells; and a combination therapeutic for the treatment of cancer comprising said peptide, fusion polypeptide, polynucleotide vector, pharmaceutical composition, immunogenic composition or vaccine.

### BACKGROUND

We have discovered novel peptides which stimulate the production of a new class of T-cells effective for treating cancer.

It is established thinking that T-cells recognise individual cancer peptides through their cognate T-cell receptor. Thus, it has been thought that a single TCR recognises a single cancer antigenic peptide typically when presented at the cell surface in the context of human leukocyte antigen (HLA) class I or class II molecule.

This new work presented herein remarkably and significantly shows some T-cells recognise different cancer antigenic peptides (of distinct sequence) using the same T-cell receptor (TCR) thus indicating that a single TCR has the ability to recognise multiple and distinct cancer antigens. This is a unique finding that goes against conventional wisdom and has significantly beneficial implications in the treatment of cancer which is thought to be a multifaceted disease.

Our work shows these T-cells can recognise multiple, distinct peptides that are derived from different cancer antigens when presented at the cell surface in the context of the same human leukocyte antigen (HLA) class I molecule. In most cases the peptides are presented at the surface of the same cancer cell, which has not been described before.

It therefore appears that some rare T-cells are capable of recognising a range of individual cancer antigenic peptides through their cognate T-cell receptor. This novel type of T-cell utilises an identical T-cell receptor (TCR) to recognise cancer cells via multiple different cancer peptides. We have termed these T-cells “multipronged T-cells” which, using their cognate TCR, can recognise and attack cancer cells via more than one antigen and thereby vastly reduce the chances of immune escape by cancer cells.

In 2015 about 90.5 million people had cancer. About 14.1 million new cases occur a year (not including skin cancer other than melanoma). It causes about 8.8 million deaths (15.7%) of human deaths. The most common types of cancer in males are lung cancer, prostate cancer, colorectal cancer, and stomach cancer. In females, the most common types of cancer are breast cancer, colorectal cancer, lung cancer and cervical cancer. If skin cancer, other than melanoma, were included in total new cancers each year it would account for around 40% of cases. In children, acute lymphoblastic leukaemia and brain tumours are most common except in Africa where non-Hodgkin lymphoma occurs more often. In 2012, about 165,000 children under 15 years of age were diagnosed with cancer. The risk of cancer increases significantly with age and many cancers occur more commonly in developed countries. Rates are increasing as more people live to an old age and as lifestyle changes occur in the developing world. The financial costs of cancer were estimated at $1.16 trillion USD per year as of 2010. It follows that there is a need to provide better and safer ways of treating or eradicating this disease. An immunotherapy that uses the body's natural defence systems to kill aberrant tissue is acknowledged to be safer than chemical intervention but, to be effective, the immunotherapy must be able to clear the disease. Moreover, the discovery of an immunotherapy that is effective against any type of cancer or a number of cancers would be extremely beneficial as not only could it be administered to individuals suffering from many different types of cancer (i.e., it would have pan-population application) but it could also be administered to a single individual suffering from more than one type of cancer.

The T-cells and their receptors we have identified herein have the afore advantageous characteristics in that they are effective against more than one type of cancer thus safeguarding against a cancer evading the effectiveness of the immune system. Further, the production of these advantageous T-cells and their receptors can be brought about by the use of the new peptides described herein.

### BRIEF DESCRIPTION OF THE INVENTION

According to a first aspect of the invention there is provided a peptide, wherein the amino acid sequence of the peptide comprises an amino acid sequence selected from the group consisting of:

(SEQ ID NO: 80)
MTSAIGILPV;
(SEQ ID NO: 77)
ITSAIGILPV;
(SEQ ID NO: 76)
ITSAIGVLPV;
(SEQ ID NO: 78)
MTSAIGVLPV;
(SEQ ID NO: 79)
QTSAIGVLPV;
(SEQ ID NO: 81)
LTSAIGVLPV;
(SEQ ID NO: 82)
ITSGIGVLPV;
(SEQ ID NO: 83)
ITSAIGVLPI;
(SEQ ID NO: 84)
QTSAIGILPV;
and
(SEQ ID NO: 85)
ITSAIGVLFV.

According to a second aspect of the invention there is provided a peptide, wherein the amino acid sequence of the peptide consists of an amino acid sequence having at least 80% sequence identity to a sequence selected from the group consisting of:

(SEQ ID NO: 76)
ITSAIGVLPV;
(SEQ ID NO: 77)
ITSAIGILPV;
(SEQ ID NO: 78)
MTSAIGVLPV;
(SEQ ID NO: 79)
QTSAIGVLPV;
(SEQ ID NO: 80)
MTSAIGILPV;
(SEQ ID NO: 81)
LTSAIGVLPV;
(SEQ ID NO: 82)
ITSGIGVLPV;
(SEQ ID NO: 83)
ITSAIGVLPI;
(SEQ ID NO: 84)
QTSAIGILPV;
and
(SEQ ID NO: 85)
ITSAIGVLFV.

### DETAILED DESCRIPTION OF THE INVENTION

Any peptide (i) the amino acid sequence of which comprises one of the aforesaid 10 peptide sequences of SEQ ID NOs 76-85, or (ii) the amino acid sequence of which consists of one of the aforesaid 10 peptide sequences of SEQ ID NOs 76-85 or an amino acid sequence having at least 80% sequence identity thereto, is referred to herein as “a peptide of the invention”.

According to some principal aspects of the invention, each peptide of the invention may be characterised as an anti-cancer peptide.

For example, the peptide has at least 90% sequence identity to a sequence selected from that group. Thus, for example, the amino acid sequence of the peptide consists of an amino acid sequence having at least 90% sequence identity to a sequence selected from the aforesaid 10 peptide sequences of SEQ ID NOs 76-85.

In an embodiment, the peptide includes one or two substitutions, deletions, or additions (such as one or two substitutions or such as terminal deletions or additions and in particular such as substitutions) relative to a sequence selected from that group. In a preferred embodiment, the peptide is identical (i.e., has 100% sequence identity) to a sequence selected from that group.

Most ideally, the amino acid sequence of said peptide is MTSAIGILPV (SEQ ID NO: 80) or an amino acid sequence having at least 80% or at least 90% sequence identify thereto, for example the amino acid sequence of said peptide is MTSAIGILPV.

In another embodiment, said the amino acid sequence of peptide is ITSAIGILPV (SEQ ID NO: 77) or an amino acid sequence having at least 80% or at least 90% sequence identify thereto, for example the amino acid sequence of said peptide is ITSAIGILPV (SEQ ID NO: 77).

Suitably, peptides of the invention which have amino acid sequences which are variants of SEQ ID NOs: 76-85 do not have insertions or deletions within the sequence. Suitably they have substitutions only. Suitably they do not have substitutions in positions 2, 3, 5, 6 and 8 of the said sequences (thus these residues are T, S, I, G and L respectively). Suitably, the possible variation in position 1 of the said sequences is M, I, Q or L and is most suitably M or I. Suitably, the possible variation in position 4 of the said sequences is A or G and is most suitably A. Suitably, the possible variation in position 7 of the said sequences is I or V and is most suitably I. Suitably, the possible variation in position 9 of the said sequences is P or F and is most suitably P. Suitably, the possible variation in position 10 of the said sequences is V or I and is most suitably V.

The invention also provides a fusion polypeptide comprising (a) two or more peptides of the invention or (b) one or more peptides of the invention and another immunogenic peptide (a “fusion polypeptide of the invention”). The fusion polypeptide may, for example comprise (a) two or three or four or five or six or seven or eight or nine or ten peptides of the invention or (b) one or two or three or four or five or six or seven or eight or nine or ten peptides of the invention and another immunogenic peptide sequence. A fusion polypeptide is a polypeptide that contains two peptide or protein sequences that are not contiguous in nature. Thus, a fusion polypeptide is an artificial sequence. Another immunogenic peptide sequence may, for example, be another sequence that stimulates the production of T-cells, especially T-cells having anti-cancer activity. Another immunogenic peptide sequence may be a peptide sequence comprising one or more peptide antigens selected from tumour-associated antigens, cancer-specific neoantigens and cancer testis antigens.

In an embodiment, said peptide or fusion polypeptide is presented by a human leukocyte antigen (HLA) class I molecule is selected from the group comprising: HLA A, HLA A2 or HLA 24 or HLA A1 or HLA A3, particularly HLA A2.

Suitably the peptide or fusion polypeptide, when administered to a subject, or ex vivo, primes the production of anti-cancer T-cells that act as effector T-cells and/or T-cells expressing said TCR that recognises a plurality of cancer antigens when said antigens are presented by the same cancer cell at a cell surface by human leukocyte antigen (HLA) class I molecule and wherein said antigens are distinct from each other and are presented by cells from different types of cancer.

Reference herein to cancer antigens that are distinct from each other is reference to cancer antigens that are representative of different types of cancer and so reference to antigens that are distinctly different in terms of their sequence structure or the molecule, typically protein, from which they are derived.

### SEQUENCE COMPARISONS

For the purposes of comparing two closely-related peptide sequences, the “% sequence identity” between a first sequence and a second sequence may be calculated. Peptide sequences are said to be the same as or identical to other peptide sequences, if they share 100% sequence identity over their entire length. Residues in sequences are numbered from left to right, i.e., from N- to C-terminus for peptides. The terms “identical” or percentage “identity”, in the context of two or more peptide sequences, refer to two or more sequences or sub-sequences that are the same or have a specified percentage of amino acid residues that are the same (i.e., at least 80% or at least 90% sequence identity over a specified region), when compared and aligned for maximum correspondence over a comparison window. Suitably, the comparison is performed over a window corresponding to the entire length of the reference sequence.

For sequence comparison, one sequence acts as the reference sequence, to which the test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percentage sequence identities for the test sequences relative to the reference sequence, based on the program parameters.

A “comparison window”, as used herein, refers to a segment in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerised implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An example of an algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. Software for performing BLAST analyses is publicly available through the National Center for Biotechnology Information (website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance.

A “difference” between sequences refers to an insertion, deletion, or substitution of a single residue in a position of the second sequence, compared to the first sequence. Two sequences can contain one or two such differences. Insertions, deletions, or substitutions in a second sequence which is otherwise identical (100% sequence identity) to a first sequence result in reduced % sequence identity. For example, if the identical sequences are 10 residues long, one substitution in the second sequence results in a sequence identity of 90% and two substitutions in the second sequence results in a sequence identity of 80%.

Alternatively, for the purposes of comparing a first, reference sequence to a second, comparison sequence, the number of additions, substitutions and/or deletions made to the first sequence to produce the second sequence may be ascertained. An addition is the addition of one residue into the first sequence (including addition at either terminus of the first sequence). A substitution is the substitution of one residue in the first sequence with one different residue. A deletion is the deletion of one residue from the first sequence (including deletion at either terminus of the first sequence).

### Polynucleotides and Vectors

According to the invention there is provided a polynucleotide encoding said peptide of the invention or fusion polypeptide of the invention.

In an embodiment, such a polynucleotide may be a chimeric polynucleotide comprising an open reading frame encoding the peptide or fusion polypeptide and a heterologous promoter and/or other transcription control element such as a terminating signal operably linked thereto.

The term “polynucleotide” refers to a polymeric macromolecule made from nucleotide monomers particularly deoxyribonucleotide or ribonucleotide monomers. The term encompasses polynucleotides containing known nucleotide analogues or modified backbone residues or linkages, which are naturally occurring and non-naturally occurring, which have similar properties as the reference polynucleotide, and which are intended to be metabolized in a manner similar to the reference nucleotides or are intended to have extended half-life in the system. Examples of such analogues include, without limitation, phosphorothioates, phosphoramidates, methyl phosphonates, chiral-methyl phosphonates, 2-O-methyl ribonucleotides, peptide-nucleic acids (PNAs). Suitably the term “polynucleotide” refers to naturally occurring polymers of deoxyribonucleotide or ribonucleotide monomers. Suitably the polynucleotides of the invention are recombinant. Recombinant means that the polynucleotide is the product of at least one of cloning, restriction or ligation steps, or other procedures that result in a polynucleotide that is distinct from a polynucleotide found in nature (e.g., in the case of cDNA). In an embodiment the polynucleotide of the invention is an artificial polynucleotide sequence (e.g., a cDNA sequence or polynucleotide sequence with non-naturally occurring codon usage). In one embodiment, the polynucleotides of the invention are DNA. Alternatively, the polynucleotides of the invention are RNA.

DNA (deoxyribonucleic acid) and RNA (ribonucleic acid) refer to polynucleotides having a backbone of sugar moieties which are deoxyribosyl and ribosyl moieties respectively. The sugar moieties may be linked to bases which are the 4 natural bases (adenine (A), guanine (G), cytosine (C) and thymine (T) in DNA and adenine (A), guanine (G), cytosine (C) and uracil (U) in RNA). As used herein, a “corresponding RNA” is an RNA having the same sequence as a reference DNA but for the substitution of thymine (T) in the DNA with uracil (U) in the RNA. The sugar moieties may also be linked to unnatural bases such as inosine, xanthosine, 7-methylguanosine, dihydrouridine and 5-methylcytidine. Natural phosphodiester linkages between sugar (deoxyribosyl/ribosyl) moieties may optionally be replaced with phosphorothioates linkages. Suitably polynucleotides of the invention consist of the natural bases attached to a deoxyribosyl or ribosyl sugar backbone with phosphodiester linkages between the sugar moieties.

In an embodiment the polynucleotide of the invention is a DNA, including single- or double-stranded DNA and straight-chain or circular DNA (i.e., plasmid DNA).

Due to the degeneracy of the genetic code, a large number of different, but functionally identical polynucleotides can encode any given peptide. For instance, the codons GCA, GCC, GCG and GCU all encode the amino acid alanine. Thus, at every position where an alanine is specified by a codon, the codon can be altered to any of the corresponding codons described without altering the encoded peptide. Such polynucleotide variations lead to “silent” (sometimes referred to as “degenerate” or “synonymous”) variants, which are one species of conservatively modified variations. Every polynucleotide sequence disclosed herein which encodes a peptide also enables every possible silent variation of the polynucleotide. One of skill will recognise that each codon in a polynucleotide (except AUG, which is ordinarily the only codon for methionine, and UGG, which is ordinarily the only codon for tryptophan) can be modified to yield a functionally identical molecule.

Accordingly, each silent variation of a polynucleotide that encodes a peptide is implicit in each described sequence and is provided as an aspect of the invention.

Degenerate codon substitutions may also be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; Rossolini et al., 1994, Mol. Cell. Probes 8:91-98).

Codons of the polynucleotide sequences of the invention may be altered in order that sequence variants of the peptide or fusion polypeptide are expressed as discussed above. In an embodiment, up to 2 codons are altered e.g., one or two e.g., one codons are altered such that a different amino acid is encoded where the codon alteration occurs. Codon alterations may involve the alteration of one, two or three bases in the polynucleotide according to the amino acid alteration to be achieved.

According to a yet further aspect of the invention there is provided a vector encoding said peptide of the invention or fusion polypeptide of the invention. There is also provided a vector comprising such a polynucleotide of the invention. Specifically, there is provided a vector for delivery of a polynucleotide to cells comprising a polynucleotide encoding the peptide of the invention or fusion polypeptide of the invention.

The vector should suitably comprise such elements as are necessary for permitting transcription of a translationally active RNA molecule in the host cell, such as a promoter and/or other transcription control elements such as an internal ribosome entry site (IRES) or a termination signal. A “translationally active RNA molecule” is an RNA molecule capable of being translated into a protein by the host cell's translation apparatus.

Example promoters to drive transcription of the peptide or fusion polypeptide include constitutive promoters such as the cytomegalovirus (CMV) promoter and elongation factor 1α (EF1α) promoter.

The vector may be, for example, a viral vector. Examples of viral vectors include vectors derived from γ-retrovirus, adenovirus, adeno-associated virus (AAV), alphavirus, lentivirus, herpes virus, arenavirus, measles virus, poxvirus or rhabdovirus.

### FORMULATION AND USE EMBODIMENTS

According to a further aspect of the invention there is provided a vaccine comprising said peptide or fusion polypeptide or vector.

According to a further aspect of the invention there is provided a pharmaceutical composition or immunogenic composition comprising said peptide, fusion polypeptide, polynucleotide encoding said peptide or fusion polypeptide or vector comprising said polynucleotide.

According to a further aspect of the invention there is provided a method of treating cancer comprising administering the peptide, fusion polypeptide, polynucleotide encoding said peptide or fusion polypeptide or vector, as such or as a vaccine, pharmaceutical composition or immunogenic composition comprising the same to a subject.

According to a further aspect of the invention there is provided a peptide, fusion polypeptide, polynucleotide encoding said peptide or fusion polypeptide or vector (or corresponding vaccine, pharmaceutical composition, or immunogenic composition for use in treating cancer. According to a further aspect of the invention there is provided the use of the peptide or fusion polypeptide (or corresponding vaccine, pharmaceutical composition, or immunogenic composition) in the manufacture of a medicament for treating cancer.

Suitably the pharmaceutical composition, immunogenic composition or vaccine is formulated under sterile conditions and is suitable for parenteral administration. For parenteral administration, the carrier preferably comprises water and may contain buffers for pH control, stabilising agents e.g., surfactants and amino acids and tonicity modifying agents e.g., salts and sugars. Pharmaceutical compositions, immunogenic compositions and vaccines may comprise immunomodulatory agents (such as adjuvants) to enhance the immune response that they raise upon administration.

### CANCER TREATMENT

Ideally said cancer that may be treated is of any type. More ideally, said cancer is selected from the group comprising or consisting of: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumour, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, ovarian, blood, colon cancer, rectal cancer, oesophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian, endocrine, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

Most preferably said cancer is pancreatic, blood, ovarian, skin, breast, bone, kidney, colon, cervical, liver, prostate or lung cancer, particularly skin cancer (e.g., melanoma), renal cell carcinoma, leukaemia, colon cancer, breast cancer or prostate cancer more particularly skin cancer (e.g., melanoma), renal cell carcinoma or leukaemia.

In a preferred embodiment of the invention said cancer is skin cancer e.g. melanoma.

### ANTIGEN POOLS AND EX VIVO STIMULATION

The invention provides an antigen pool comprising two or more (e.g., 2, 3, 4, 5, 6, 7, 8, 9 or 10) peptides of the invention.

In an embodiment, the invention provides an antigen pool antigen pool comprising of two or more peptides, said peptides having an amino acid sequence selected from:

(a) the amino acid sequence consisting of MTSAIGILPV (SEQ ID NO: 80) or an amino acid sequence having at least 80% sequence identity thereto, or an amino acid sequence comprising the sequence of MTSAIGILPV (SEQ ID NO: 80);

(b) the amino acid sequence consisting of ITSAIGILPV (SEQ ID NO: 77) or an amino acid sequence having at least 80% sequence identity thereto, or an amino acid sequence comprising the sequence of ITSAIGILPV (SEQ ID NO: 77);

(c) the amino acid sequence consisting of ITSAIGVLPV (SEQ ID NO: 76) or an amino acid sequence having at least 80% sequence identity thereto, or an amino acid sequence comprising the sequence of ITSAIGVLPV (SEQ ID NO: 76);

(d) the amino acid sequence consisting of MTSAIGVLPV (SEQ ID NO: 78) or an amino acid sequence having at least 80% sequence identity thereto, or an amino acid sequence comprising the sequence of MTSAIGVLPV (SEQ ID NO: 78);

(e) the amino acid sequence consisting of QTSAIGVLPV (SEQ ID NO: 79) or an amino acid sequence having at least 80% sequence identity thereto, or an amino acid sequence comprising the sequence of QTSAIGVLPV (SEQ ID NO: 79);

(f) the amino acid sequence consisting of LTSAIGVLPV (SEQ ID NO: 81) or an amino acid sequence having at least 80% sequence identity thereto, or an amino acid sequence comprising the sequence of LTSAIGVLPV (SEQ ID NO: 81);

(g) the amino acid sequence consisting of ITSGIGVLPV (SEQ ID NO: 82) or an amino acid sequence having at least 80% sequence identity thereto, or an amino acid sequence comprising the sequence of ITSGIGVLPV (SEQ ID NO: 82);

(h) the amino acid sequence consisting of ITSAIGVLPI (SEQ ID NO: 83) or an amino acid sequence having at least 80% sequence identity thereto, or an amino acid sequence comprising the sequence of ITSAIGVLPI (SEQ ID NO: 83);

(i) the amino acid sequence consisting of QTSAIGILPV (SEQ ID NO: 84) or an amino acid sequence having at least 80% sequence identity thereto, or an amino acid sequence comprising the sequence of QTSAIGILPV (SEQ ID NO: 84); and

(j) the amino acid sequence consisting of ITSAIGVLFV (SEQ ID NO: 85) or an amino acid sequence having at least 80% sequence identity thereto, or an amino acid sequence comprising the sequence of ITSAIGVLFV (SEQ ID NO: 85).

The peptides of the invention and pools containing them may be used as antigens to stimulate or amplify T-cells ex vivo.

Suitably antigen pools comprise at least (i) a peptide having an amino acid sequence consisting of the amino acid sequence TSAIGILPV (SEQ ID NO: 80) or an amino acid sequence having at least 80% sequence identity thereto and/or (ii) a peptide having an amino acid sequence consisting of the amino acid sequence ITSAIGILPV (SEQ ID NO: 77) or an amino acid sequence having at least 80% sequence identity thereto.

Alternatively, suitably antigen pools comprise at least (i) a peptide having an amino acid sequence comprising the amino acid sequence TSAIGILPV (SEQ ID NO: 80) and/or (ii) a peptide having an amino acid sequence comprising the amino acid sequence ITSAIGILPV (SEQ ID NO: 77).

Fusion polypeptides may also be used as antigens to stimulate or amplify T-cells ex vivo.

Thus, the invention provides use of one or more peptides of the invention or one or more fusion polypeptides of the invention as antigens, or the aforementioned antigen pool, in the ex vivo stimulation and/or amplification of T-cells derived from a human suffering from cancer, in particular, for subsequent reintroduction of said stimulated and/or amplified T-cells into the said human for the treatment of the said cancer in the said human.

The invention provides an ex vivo method of stimulating and/or amplifying T-cells which comprises contacting said T-cells, optionally together with antigen-presenting cells, with one or more peptides of the invention or one or more fusion polypeptides of the invention as antigens or with the aforementioned antigen pool. The T-cells optionally together with antigen-presenting cells may suitably be obtained from a cancer patient (i.e., suitably are autologous T-cells and antigen-presenting cells).

In an embodiment, T-cells are contacted with antigen-presenting cells which present peptides of the invention (or any one of them) in complex with HLA peptides, particularly HLA Class I peptides.

The invention provides a population of stimulated and/or amplified T-cells obtainable or obtained according to this method. The invention provides an antigen-presenting cell which presents a peptide of the invention in complex with an HLA peptide, such as an HLA Class I peptide.

The invention provides a method of treatment of cancer in a human which comprises taking from said human T-cells optionally together with antigen-presenting cells, stimulating and/or amplifying said T-cells in the presence of one or more peptides of the invention or one or more fusion polypeptides of the invention, or the aforementioned antigen pool, and reintroducing some or all of said stimulated and/or amplified T-cells into the human.

The stimulation and/or amplification of T-cells may be enhanced by use of an immune stimulant (i.e., an adjuvant) together with the one or more peptides of the invention or one or more fusion polypeptides of the invention.

The one or more peptides or one or more fusion polypeptides may be provided as polynucleotides or vectors as described herein such that one or more peptides of the invention or one or more fusion polypeptides of the invention are expressed in the presence of the T-cells.

### TCRS

According to a further aspect of the invention there is provided an isolated T-cell receptor (TCR), or a fragment thereof, that recognises a plurality of cancer peptide antigens when said antigens are presented at a cell surface by human leukocyte antigen (HLA) class I molecule and wherein said antigens are distinct from each other and are representative of more than one type of cancer.

According to a further aspect of the invention there is provided an TCR or a cancer specific TCR, or a fragment thereof, that recognises a plurality of cancer antigens wherein said TCR has a complementarity-determining region selected from the group comprising or consisting of:

(SEQ ID NO: 1)
CATSDRGQGANWDEQFF;
(SEQ ID NO: 2)
CASTLGGGTEAFF;
(SEQ ID NO: 3)
CSARDLLAETYEQYF;
(SEQ ID NO: 4)
CASSSSDTDTQYF;
(SEQ ID NO: 5)
CSVEGSLGRALRANEQFF;
(SEQ ID NO: 6)
CATHGGEKLFF;
(SEQ ID NO: 7)
CASSYVGLGSPLHF;
(SEQ ID NO: 8)
CSGQANTEAFF;
(SEQ ID NO: 9)
CASSPTTGLKTRSGYTF;
(SEQ ID NO: 10)
CSEGSPYNEQFF;
(SEQ ID NO: 11)
CASSNGFHFNTLYF;
(SEQ ID NO: 12)
CASSLGGGDTQYF;
(SEQ ID NO: 13)
CASSFAGTDTQYF;
(SEQ ID NO: 14)
CASSLGEGSPGELFF;
(SEQ ID NO: 15)
CASSQEPNWNTEAFF;
(SEQ ID NO: 16)
CASSFQGPGYGYTF;
(SEQ ID NO: 17)
CSARDTTWGLEQYF;
(SEQ ID NO: 18)
CATKPSGSTDTQYF;
(SEQ ID NO: 19)
CSARDEGIGYEQYF;
(SEQ ID NO: 20)
CASSSGPGELFF;
(SEQ ID NO: 21)
CARRTLVIVRRFYSGNTIYF;
(SEQ ID NO: 22)
CSARDLIGSQTYEQYF;
(SEQ ID NO: 23)
CSARDPIGTESYEQYF;
(SEQ ID NO: 24)
CSARDRAGRSPLHF;
(SEQ ID NO: 25)
CSVEESSGIYEQYF;
(SEQ ID NO: 26)
CSAREDGGQTYEQYF;
(SEQ ID NO: 27)
CASSWAGPVEQYF;
(SEQ ID NO: 28)
CASSSQGRAEQYF;
(SEQ ID NO: 29)
CASSSRDSLYEQYF;
(SEQ ID NO: 30)
CASSLGIISGQPQHF;
(SEQ ID NO: 31)
CASSNTGGYTQYF;
(SEQ ID NO: 32)
CASSQGLLLDNEQFF;
(SEQ ID NO: 33)
CASSSPMDSGDTDTQYF;
(SEQ ID NO: 34)
CASSPRSGVPQHF;
(SEQ ID NO: 35)
CASSFVREEGSTDTQYF;
(SEQ ID NO: 36)
CSARGTESYEQYF;
(SEQ ID NO: 37)
CASWPGEGFGETQYF;
(SEQ ID NO: 38)
CSGWGQGDEKLFF;
(SEQ ID NO: 39)
CASSEYTSGNQPQHF;
(SEQ ID NO: 40)
CSARDLVVTGETYEQYF;
(SEQ ID NO: 41)
CSATGLAGLGEQFF;
(SEQ ID NO: 42)
CATSDLGTGVGEQFF;
(SEQ ID NO: 43)
CSVGPGSTGELFF;
(SEQ ID NO: 44)
CASSPTGEKLFF;
(SEQ ID NO: 45)
CASSQEGGTWGDGYTF;
(SEQ ID NO: 46)
CATSDLLLAGGRSSYNEQFF;
(SEQ ID NO: 47)
CASSEAASGRPQTF;
(SEQ ID NO: 48)
CATSDATAGTSGSLYEQYF;
(SEQ ID NO: 49)
CASSLTGLGQPQHF;
(SEQ ID NO: 50)
CASSPAVLSYEQYF;
(SEQ ID NO: 51)
CSARESLAETYEQYF;
(SEQ ID NO: 52)
CASSPGLTANVLTF;
(SEQ ID NO: 53)
CASSLGLAGNEQYF;
(SEQ ID NO: 54)
CASSNGFHFNTQYF;
(SEQ ID NO: 55)
CASSLGILTDTQYF;
(SEQ ID NO: 56)
CASSFQPVDTQYF;
(SEQ ID NO: 57)
CSASEGIGQPQHF;
and
(SEQ ID NO: 58)
CASSVSGGEQFF;

or a complementarity-determining region that has at least 85% identity with any one or more of the afore complementarity-determining regions.

In a further preferred embodiment said complementarity-determining region has at least 86, 87, 88, 89, 90, 91, 92, 93, 94, 95, 96, 97, 98 or 99% identity with any one or more of the afore complementarity-determining regions.

In a preferred embodiment of the invention said plurality of antigens are presented at the cell surface in the context of human leukocyte antigen (HLA) class I molecule and, more preferably still, said recognition occurs or is shown to occur by any one or more of, including any combination of, the following activities:

said TCR, or a T cell expressing said TCR, triggers or causes death of a cancer cell expressing any one or more of said antigens; and/or

said TCR, or a T cell expressing said TCR, triggers the production of or makes pro-inflammatory cytokines such as TNF and IFN gamma (this feature is useful for reversing the immunosuppressive tumour microenvironment); and/or

said TCR, or a T cell expressing said TCR, triggers degranulation or undergoes degranulation; and/or

said TCR, or a T cell expressing said TCR, upregulates any one or more of CD107a, Beta-chemokines (MIP 1beta) and cytokines such as Interferon gamma (IFNgamma) and tumour necrosis factor (TNF).

In a preferred embodiment of the invention said TCR has a complementarity-determining region selected from the group comprising or consisting of:

(SEQ ID NO: 59)
CATSDRGQGANWDEQFF;
(SEQ ID NO: 60)
CASTLGGGTEAFF;
(SEQ ID NO: 61)
CSARDLLAETYEQYF;
(SEQ ID NO: 62)
CASSSSDTDTQYF;
(SEQ ID NO: 63)
CSVEGSLGRALRANEQFF;
(SEQ ID NO: 64)
CATHGGEKLFF;
(SEQ ID NO: 65)
CASSYVGLGSPLHF;
(SEQ ID NO: 66)
CSGQANTEAFF;
(SEQ ID NO: 67)
CASSPTTGLKTRSGYTF;
(SEQ ID NO: 68)
CSEGSPYNEQFF;
(SEQ ID NO: 69)
CASSNGFHFNTLYF;
and
(SEQ ID NO: 70)
CASSLGGGDTQYF;

or a complementarity-determining region that has at least 85% sequence identity with any one or more of the afore complementarity-determining regions.

In a further preferred embodiment said complementarity-determining region has at least 86, 87, 88, 89, 90, 91, 92, 93, 94, 95, 96, 97, 98 or 99% sequence identity with any one or more of the afore complementarity-determining regions. Sequence identity may be determined as set forth in the “Sequence comparisons” section above

In a preferred embodiment of the invention said more than one types of cancer are as set forth in the “Cancer treatment” section above.

Reference herein to cancer antigens that are distinct from each other is reference to cancer antigens that are representative of different types of cancer and so reference to antigens that are distinctly different in terms of their sequence structure or the molecule, typically protein, from which they are derived.

Nevertheless, despite this difference in antigen sequence the TCR of the invention is able to recognise a plurality of these distinct or different cancer antigens. Those skilled in the art will appreciate, it would be extremely difficult for cancer cells to escape from T-cells that were targeting them through more than one different cancer antigen as escape would require simultaneous mutation of all targets that lowered or ablated presentation of all cognate peptides.

In a preferred embodiment of the invention said human leukocyte antigen (HLA) class I molecule is MHC class I (A, B, or C). More specifically, said HLA is HLA A2 or HLA A24 or HLA A1 or HLA A3.

MHC class I present peptides from inside the cell. For example, in the context of a cancer cell, the HLA system brings fragments or peptides of the cancer-expressed protein to the surface of the cell so that the cell can be recognised as cancerous and destroyed by the immune system. These peptides are produced from digested proteins that are broken down in the proteasomes. In general, these particular peptides are small polymers, about 7-20, typically but not exclusively 9 or 10 amino acids in length. Oncogenic antigens presented by MHC class I system attract killer T-cells (also called CD8 positive- or cytotoxic T-cells) that destroy the cancer cells.

In a preferred embodiment of the invention said TCR is an alpha beta (αβ) TCR.

In yet a further preferred embodiment, said TCR is a soluble TCR (sTCR) and so lacks the transmembrane and, ideally also, intracellular domains.

In yet another preferred embodiment of the invention said TCR is part of a chimeric receptor having the functionality described herein. Ideally, said TCR is fused to a TCR constant domain or a TCR signalling domain.

In the alternative, there is provided a fragment of said TCR such as a monomeric part thereof, ideally a single chain form of the TCR.

In a further alternative, there is provided a fragment of said TCR such as the complementarity determining region thereof.

According to a further aspect of the invention there is provided a T-cell expressing said TCR of the invention, ideally, in either a soluble form or membrane compatible form i.e., having a transmembrane region and intracellular region.

According to a yet further aspect of the invention there is provided a T-cell clone expressing said TCR of the invention, ideally, in either a soluble form and so lacks a transmembrane domain and, ideally also, an intracellular domain or a membrane compatible form i.e., having a transmembrane region and, ideally also, an intracellular domain.

Preferably said clone is a T-cell clone CR24, GD1, GD2, VB6G4.24, CR1 or VB10 as described herein.

Ideally, said clone is CR24 which recognises multiple antigenic cancer peptides, most preferably clone CR24 recognises a plurality of said peptides selected from the group comprising or consisting of: EAAGIGILTV (SEQ ID NO: 71) from Melan A (residues 26-35), LLLGIGILVL (SEQ ID NO: 72) from BST2 (residues 22-31) and NLSALGIFST (SEQ ID NO: 73) from IMP2 (residues 367-376). Preferably, this recognition is in the context of HLA A2 presentation.

Ideally, said clone GD1 or GD2 recognises multiple antigenic cancer peptides, most preferably clone GD1 or GD2 recognises the following peptides: RLVDDFLLV (SEQ ID NO: 74) from human telomerase reverse transcriptase (hTERT) (residues 855-873) and ALKDVEERV (SEQ ID NO: 75) from melanoma associated antigen C2 (MAGE C2) (residues 336-344). Clone GD1 was able to kill breast, blood, and melanoma cancer cell lines.

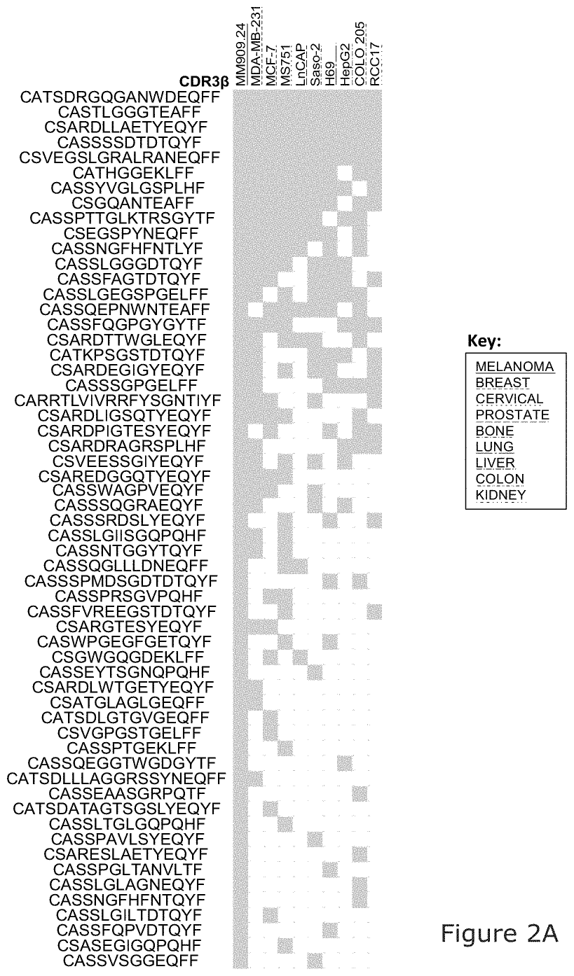

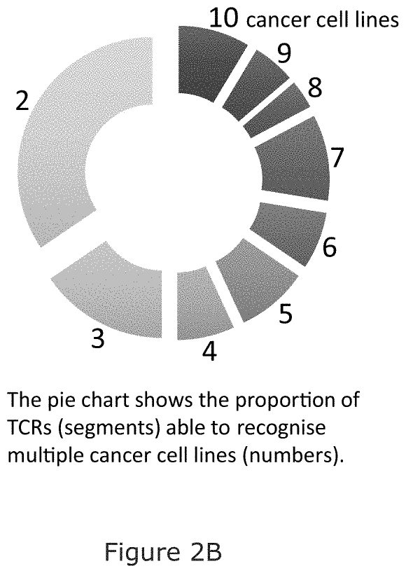

Ideally, said clones VB6G4.24, CR1 and VB10 recognise the Melan A peptide (EAAGIGILTV (SEQ ID NO: 71)) but not BST2 (LLLGIGILVL (SEQ ID NO: 72) or IMP2 (NLSALGIFST (SEQ ID NO: 73)) peptides (neither as exogenous peptide nor from transduced protein expressed by MOLT3s). Since the CDR3 sequence of the beta TCR chain from VB6G4.24 appeared in clonotyping data for all ten cancer cell lines in FIG. 2, this clone responds to multiple cancer cells lines but not by recognition of the IMP2 or BST2 peptides.

According to a yet further aspect of the invention there is provided a vector encoding said TCR of the invention.

Said TCR may form part of a bispecific antibody wherein said bispecific includes said TCR, for the purpose of binding to its ligand on a cancer cell, and also an immune cell activating component or ligand that binds and so activates an immune cell such as a Killer T-cell.

According to a yet further aspect of the invention there is provided a pharmaceutical composition or immunogenic composition or bispecific or vaccine comprising said TCR or T-cell or T-cell clone or vector of the invention.

In a preferred embodiment said pharmaceutical composition or immunogenic composition or bispecific or vaccine is for use in the treatment of cancer.

According to a yet further aspect of the invention there is provided the TCR or T-cell or T-cell clone or vector as disclosed herein for use in the treatment of cancer.

According to a yet further aspect of the invention there is provided a method of treating cancer in an individual having or suspected of having cancer comprising administering said TCR or T-cell or T-cell clone or vector or pharmaceutical composition or immunogenic composition or bispecific or vaccine to the individual to be treated.

In a preferred method of the invention said TCR, cell, clone or vector is administered in combination with an anti-cancer agent such as, but not limited to, a bispecific antibody.

Reference herein to a bispecific is reference to a bispecific monoclonal antibody (BsMAb, BsAb) which is an artificial protein that can simultaneously bind to two different types of antigen.

According to a yet further aspect of the invention there is provided the use of said TCR or cell or clone or vector in the manufacture of a medicament to treat cancer.

According to a yet further aspect of the invention there is provided a combination therapeutic for the treatment of cancer comprising:

a) said TCR or cell or clone or vector or immunogenic composition or bispecific or vaccine in combination with

b) a further cancer therapeutic agent.

According to a yet further aspect of the invention there is provided a peptide or peptide antigen able to elicit anti-cancer T-cells, which, ideally but not exclusively, recognises said TCR of the invention, or a part thereof, and which when administered to a subject primes the production of: anti-cancer T-cells that act as effector T-cells and/or T-cells that recognise a plurality of cancer antigens when said peptide antigens are presented at a cell surface by human leukocyte antigen (HLA) class I molecule and wherein said cancer antigens are distinct from each other and are representative of more than one type of cancer.

In the claims which follow and in the preceding description of the invention, except where the context requires otherwise due to express language or necessary implication, the word “comprises”, or variations such as “comprises” or “comprising” is used in an inclusive sense i.e., to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds, or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with reference to the following wherein:

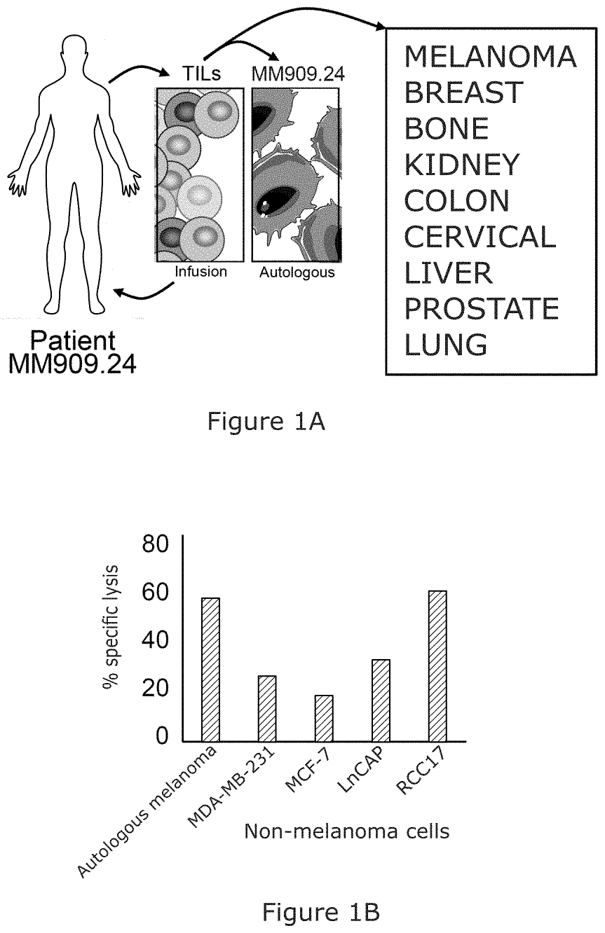

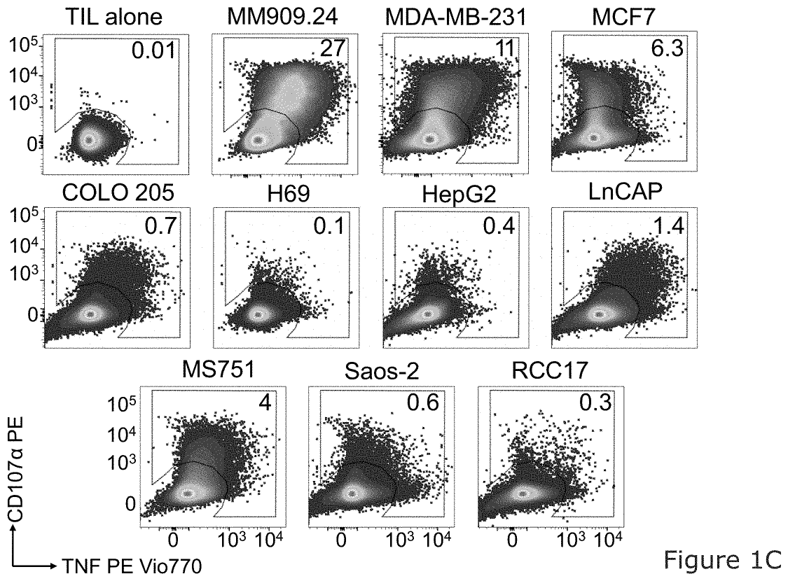

FIGS. 1A-C show tumour infiltrating lymphocytes (TILs) used to cure HLA A2+ patient MM909.24 of metastatic melanoma are capable of recognising multiple HLA A2+ cancer cell types. (FIG. 1A) The TILs were tested against autologous melanoma and cancer cell lines of different tissue origin. (FIG. 1B) Chromium release cytotoxicity assay with autologous melanoma and the HLA A2+ cancer cell lines displayed. Specific lysis after 18 h of incubation is displayed. (FIG. 1C) TAPI-0 assay whereby TILs were incubated with the indicated HLA A2+ cancer cell lines for 5 h and activation assessed by detection of TNF and CD107a with monoclonal antibodies. The activated gate (TNF+ and/or CD107a+) was set based on the TIL alone control. Responding T-cells were sorted by flow cytometry and used for next generation sequencing of the α and β chains of the T-cell receptor (TCR).

FIGS. 2A-B show T-cell receptor (TCR) β chains clonotypes of functional T-cells, from the TIL of HLA A2+ patient MM909.24, able to respond to cancer cell lines as well as autologous melanoma (MM909.24). Cells were sorted based on function (TAPI-0 assay with CD107a and TNF antibodies) following 5 h of incubation with the HLA A2+ cancer cell lines shown (FIG. 1) and used for high throughout IIlumina sequencing of the TCR chains. (FIG. 2A) The TCR β chain CDR3s are displayed on the left, with each shaded segment of the chart indicating that the CDR3 was present in the population responding to the cancer cell line shown at the top of the chart. Five TCRs are seen to respond to all cancers. (FIG. 2B) Shows the proportion of CDR3s that recognised the number of cancer cell lines shown next to each segment. For example, 2 cell lines=autologous melanoma+one other cancer cell line; 10 cell lines=autologous melanoma+9 other cancer cell lines. Over 50% of the clonotypes that respond to HLA A2+ autologous melanoma also respond to 4 or more other cancer types.

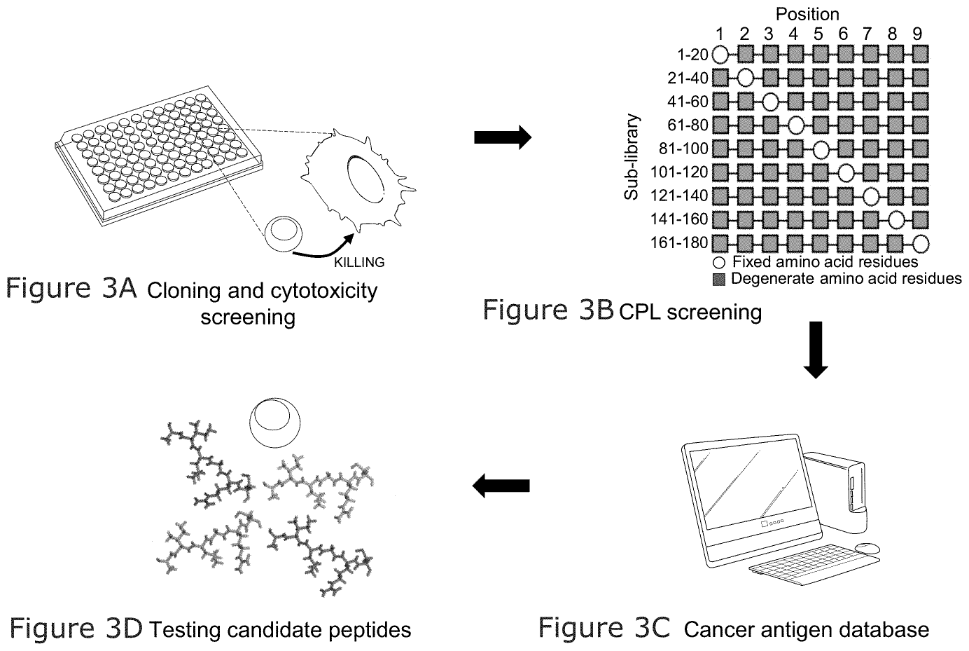

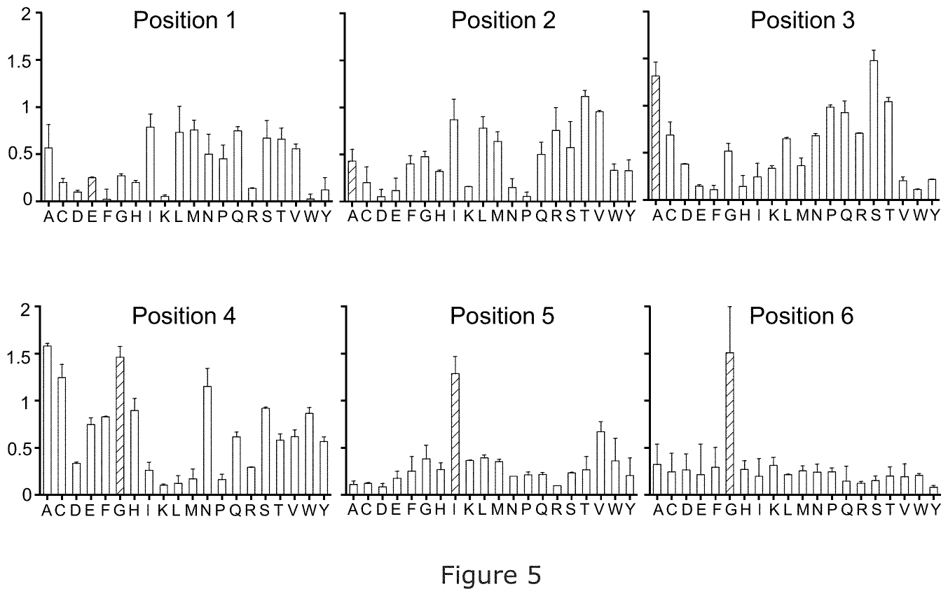

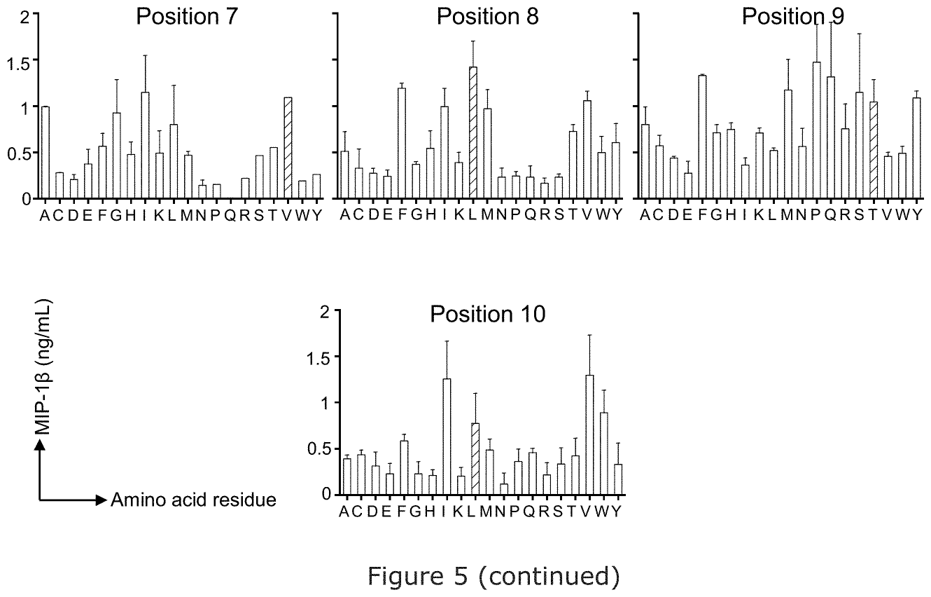

FIGS. 3A-D show a cancer epitope discovery pipeline. These figures depicts the strategy used to discover the peptide(s) recognised by T-cells that respond to multiple cancer cell types. (FIG. 3A) CD8 T-cells were cloned from TIL MM909.24 by limiting dilution then screened for cytotoxicity against autologous MM909.24 melanoma. In some cases, other cancer cell types were also used during the screening. Clones of interest were expanded and used for further assays. (FIG. 3B) Combinatorial peptide library screening was performed for key CD8 T-cell clones to reveal their amino acid residue preferences at each position of a peptide. The schematic shows the design of a CPL library, comprised of peptide sub-libraries; each sub-library has a fixed amino acid residue (open circle) (1 of the 20 proteogenic amino acids) at a defined position of the peptide, with all other positions of the same sub-library being a random mix of residues (grey square). (FIG. 3C) The CPL data (example shown in FIG. 5) was used to screen a cancer protein database (manuscript in preparation) to shortlist candidate peptides that are predicted to be recognised by the clone. (FIG. 3D) Functional testing of candidate cancer peptides to reveal those recognised by a CD8 clone.

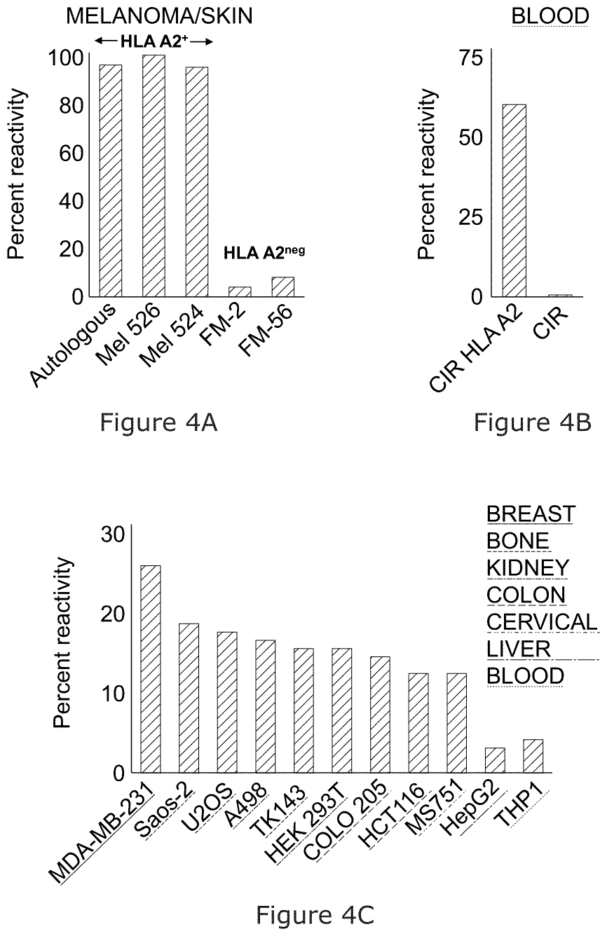

FIGS. 4A-C show T-cell cone CR24 can recognise multiple HLA A2+ cancer cell lines of different tissue origin. TAPI-0 assays were used to assess the reactivity of CR24 towards the cancer cell lines shown. The percentage of reactivity (CD107a+ and/or TNF+) is displayed. (FIG. 4A) CR24 recognised HLA A2+ melanomas but not HLA A2− negative melanomas. (FIG. 4B) The leukaemic cell line CIR was recognised when HLA A2 was expressed. (FIG. 4C) Recognition of non-melanoma HLA A2+ cell lines of different tissue origin (key).

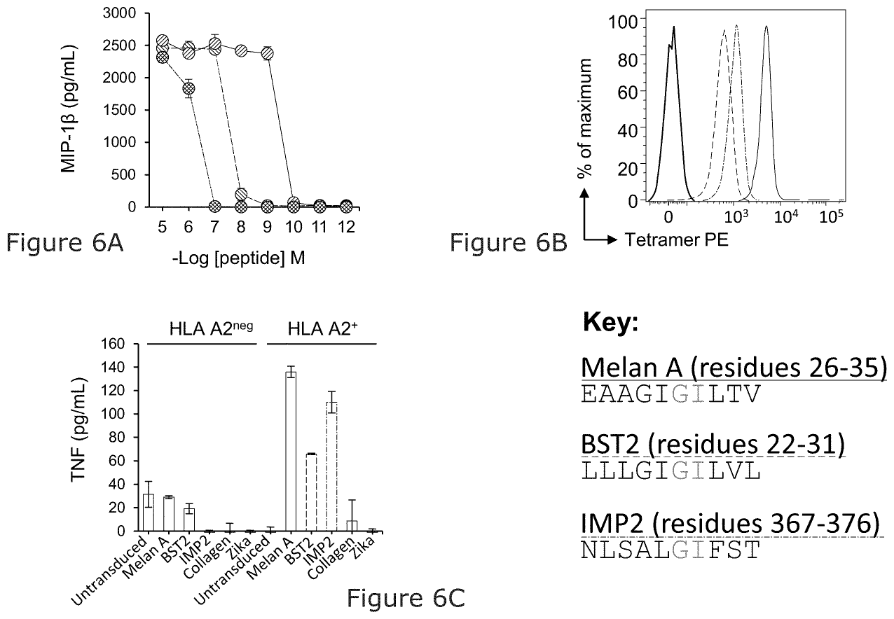

FIG. 5 shows combinatorial peptide library (CPL) screen of CD8 T-cell clone CR24. Each sub library of a decamer CPL screen was incubated in duplicate with CR24, with the TAP (transporter associated with antigen processing) deficient cell line T2 used as an antigen presenting cell. The peptide length (10mers) preference of CR24 had already been determined using a sizing scan assay (data not shown). After overnight incubation the supernatants were harvested, and clone activation assessed by MIP1-β ELISA. Each graph shows one peptide position of the CPL screen, with the amino acids (single letter code) shown on the x-axis fixed at that particular position. The bars in green show the amino acid residues for one of the peptides recognised by CR24, EAAGIGILTV from Melan A (residues 26-35). The CPL data was run via a bespoke cancer antigen webtool to give candidate peptides that are most likely to be recognised by CR24 (FIG. 6).

FIGS. 6A-C show T-cell clone CR24 recognises three distinct peptides derived from different cancer proteins. Of the candidate peptides identified by the combinatorial peptide library screen performed in FIG. 4, three of the peptides were recognised by CR24; EAAGIGILTV (Melanoma Antigen Recognised by T-cells 1/Melanocyte Antigen (MART-1/Melan A, residues 26-35) http://www.iedb.org/epld/10987), LLLGIGILVL (Bone marrow stromal antigen 2 (BST2, residues 22-31) and NLSALGIFST from Insulin-like growth factor 2 mRNA binding protein 2 (IMP2, residues 367-376). The two amino acid residues common to all three peptides are shown in grey in the key. The Melan A peptide is well described as a target of T-cells recognising melanomas. A 9-amino acid length version of the BST2 peptide has been described previously (10: https://www.ncbi.nlm.nih.gov/pubmed//16569595). The IMP2 peptide is a new epitope that has not previously been described. (FIG. 6A) Activation assay with CR24 and a titration of each peptide, incubated overnight and supernatants used for MIP-1β ELISA. (FIG. 6B) CR24 stained with HLA A2 tetramers for each of the peptides confirming that the cognate TCR could engage these antigens. An optimised staining protocol was used. The control tetramer is HLA A2 ALWGPDPAAA (preproinsulin residues 15-24). (FIG. 6C) Activation assays with CR24 and antigen presenting cells expressing the proteins that the three cancer peptides are derived from. The cell line, MOLT3 (naturally HLA-A2 negative, Melan A negative, BST2 negative and IMP2 negative) was transduced with genes for expression of HLA A2, Melan A, BST2, IMP2, the α2 subunit of collagen type IV and the anchor capsid protein from Zika virus. The collagen and Zika proteins acted as transduction/irrelevant protein controls. CR24 was incubated overnight with each of the MOLT3 cell lines and supernatants harvested for TNF ELISA.

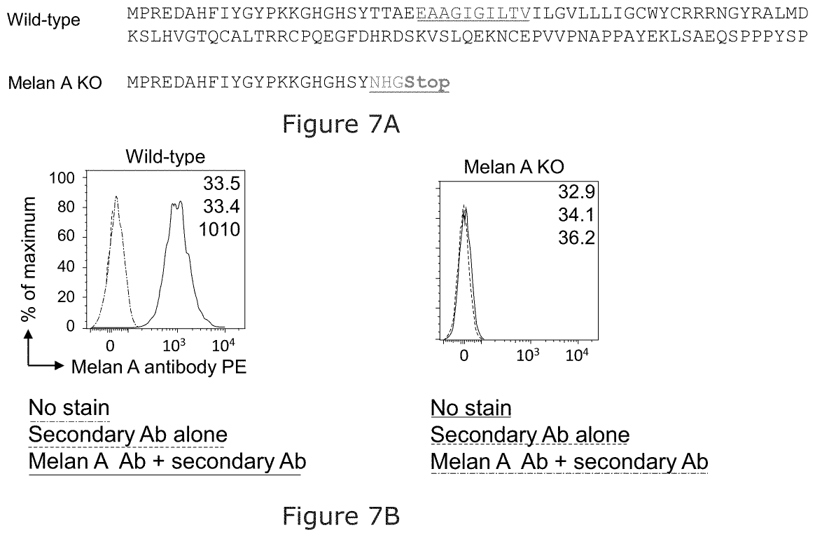

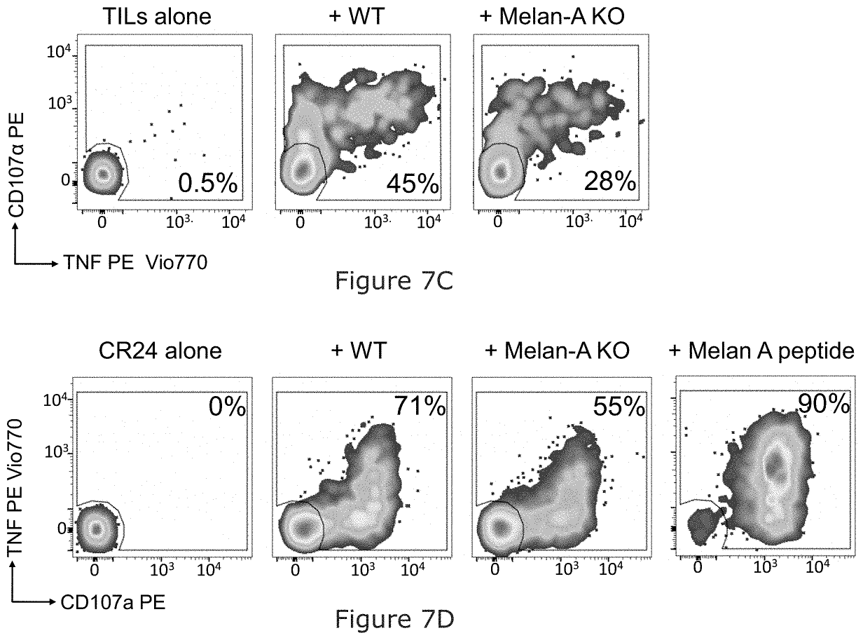

FIGS. 7A-D show T-cell clone CR24 recognises autologous melanoma through at least two antigens. (FIG. 7A) The Melan A gene in autologous MM909.24 melanoma was targeted for ablation using a guide (g) RNA and CRISPR-Cas9. The wild-type Melan A amino acid sequence is shown with the EAAGIGILTV (SEQ ID NO: 71) peptide in blue. Sequencing of the Melan A loci confirmed gene disruption due to an early STOP codon, at both alleles, which was downstream of the EAAGIGILTV (SEQ ID NO: 71) sequence. (FIG. 7B) Intracellular staining for Melan A with an unconjugated anti-Melan A antibody and PE conjugated secondary antibody confirmed the absence of Melan A protein. (C&D) Activation assays (TAPI-0 with TNF and CD107a antibodies) of TIL MM909.24 (FIG. 7C) and CR24 (FIG. 7D) with wild-type and Melan A knock-out (KO) autologous melanomas. Melan A peptide EAAGIGILTV (SEQ ID NO: 71) was used as a positive control for CR24. CR24 was still capable of recognising autologous melanoma lacking Melan A expression, and therefore HLA A2-EAAGIGILTV (SEQ ID NO: 71) presentation, suggesting that at least one other peptide was being recognised by CR24, and most likely those derived from BST2 and/or IMP2.

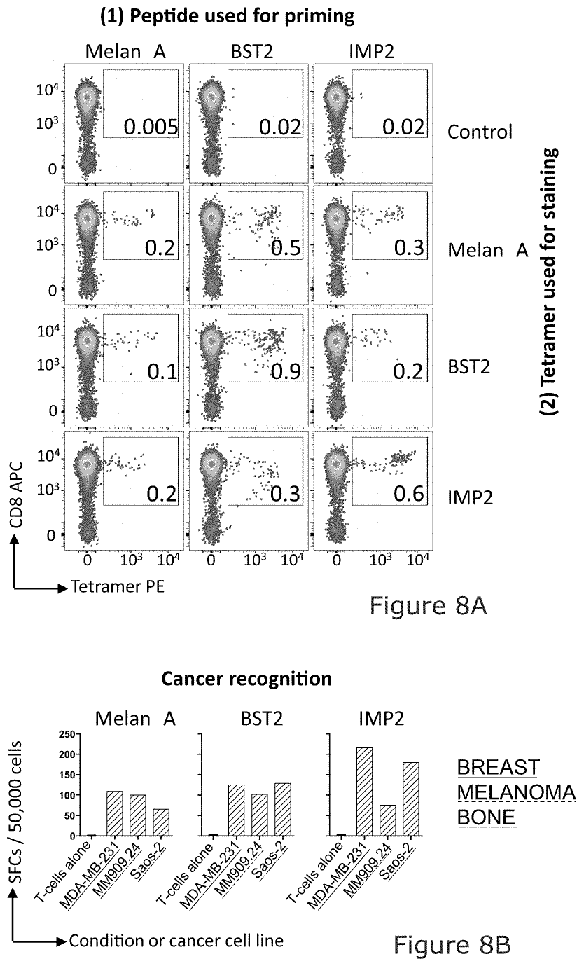

FIGS. 8A-B show T-cells cross-reactive for Melan A (EAAGIGILTV (SEQ ID NO: 71)), BST2 (LLLGIGILVL (SEQ ID NO: 72)) and IMP2 (NLSALGIFST (SEQ ID NO: 73)) peptides can be generated from healthy donor(s). (FIG. 8A) CD8 T-cells from two HLA A2+ donors (representative data from one donor is shown) were primed as separate cultures with Melan A, BST2 or IMP2 peptide (1). Two weeks post priming each culture was stained with control (ALWGPDPAAA (SEQ ID NO: 86) from preproinsulin 15-24), Melan A, BST2 and IMP2 tetramers (2). The percentage of cells staining is shown for each sample. (FIG. 8B) Each of the primed T-cell lines was used in overnight IFNy ELISpot assay with the cancer cell lines; MDA-MB-231 (breast), MM909.24 (melanoma) and Saos-2 (bone). T-cells were also incubated alone. The number of spot forming cells (SFCs) per 50,000 cells is shown.

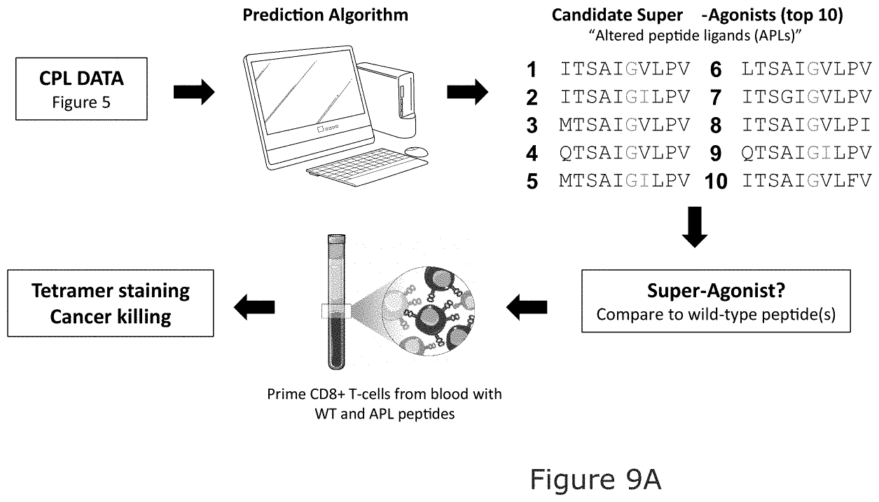

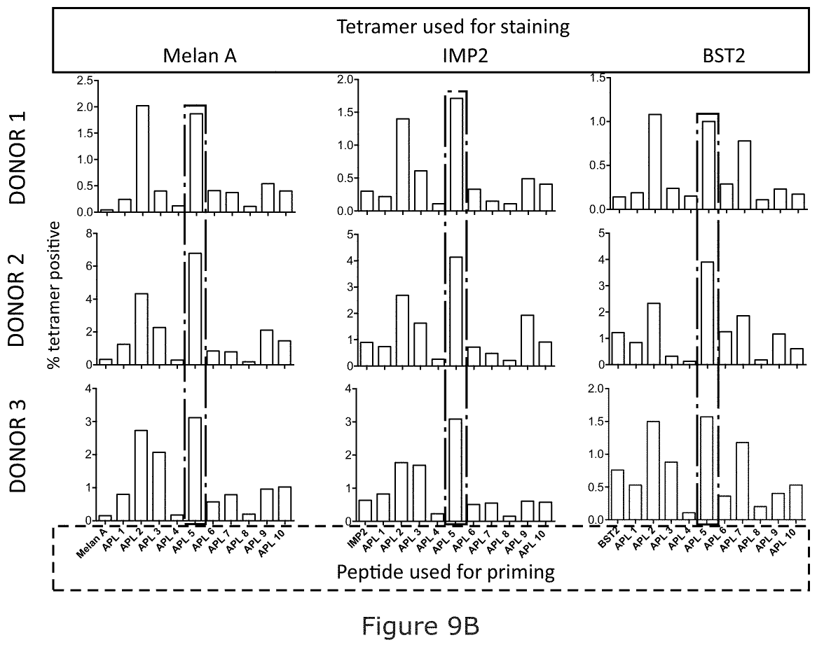

FIGS. 9A-B show that super-agonist peptide for multi-pronged T-cells primes more cancer-peptide specific T-cells than the wild-type peptides. Candidate super-agonists were designed using CPL data for CR24 (FIG. 5) and a prediction algorithm (http://wsbc.warwick.ac.uk/wsbcToolsWebpage/user_cases.php); which identifies the peptides most likely to act as a super-agonist based on the amino acid preferences revealed by the CPL data (2: https://www.ncbi.nlm.nih.gov/pubmed/22952231). The peptides are sequence dissimilar to the wild-type peptide and termed altered peptide ligands. The top ten peptides are shown in (FIG. 9A) and share either a Glycine at position 6 (Altered peptide ligands (APL), 1, 3, 4, 6, 7, 8 and 10) or Glycine and Isoleucine at positions 6 and 7 respectively (APL peptides 2, 5 and 9), with wild-type peptides EAAGIGILTV (SEQ ID NO: 71) (Melan A), LLLGIGILVL (SEQ ID NO: 72) (BST2) and NLSALGIFST (SEQ ID NO: 73) (IMP2) (shown in bold). (FIG. 9B) To test the APLs for super-agonist properties each of the WT and APL peptides were used to prime CD8+ T-cells from HLA A2+ healthy donors. The magnitude of the response to each of the peptides was assessed by staining the T-cells with tetramers for HLA A2-EAAGIGILTV (Melan A) (SEQ ID NO: 71), -LLLGIGILVL (SEQ ID NO: 72) (BST2) or -NLSALGIFST (SEQ ID NO: 73) (IMP2). Overall, APL 5 (MTSAIGILPV) (SEQ ID NO: 80) seemed to be the most effective super-agonist at priming Melan A, BST2 and IMP2 T-cells across all three donors tested, with APL 2 (ITSAIGILPV) (SEQ ID NO: 77) also exhibiting effect across each donor.

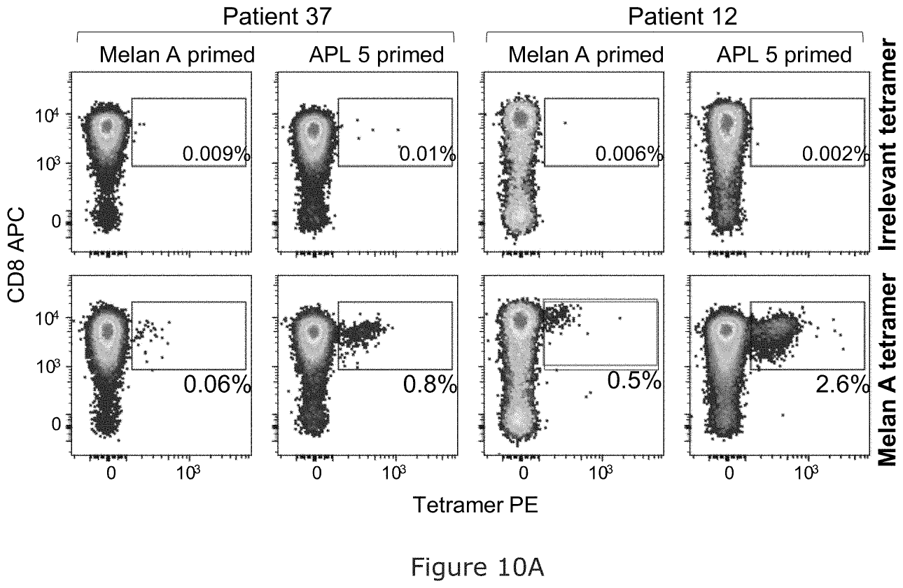

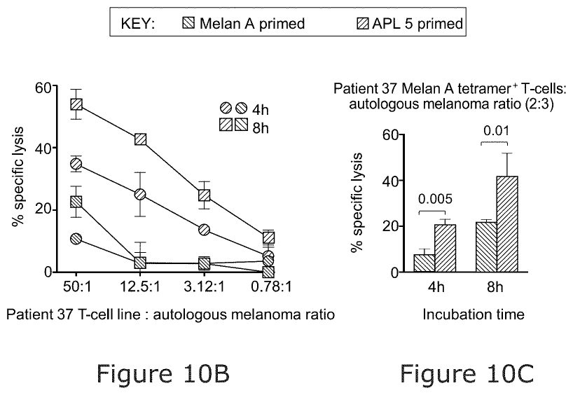

FIGS. 10A-C show that super-agonist peptide number 5 (MTSAIGILPV) (SEQ ID NO: 80) primed more CD8 T-cells from metastatic melanoma patients able to recognise WT EAAGIGILTV Melan A peptide (SEQ ID NO: 71). Due to the limited number of PBMCs available from patients 37 and 12 only the Melan A peptide was used for comparison to peptide number 5. Patient 37 is now deceased having not responded to conventional or TIL therapy. Patient 12 was undergoing therapy. (FIG. 10A) HLA A2-EAAGIGILTV (WT Melan A) (SEQ ID NO: 71) tetramer staining data following priming of CD8+ T-cells with EAAGIGILTV (WT) (SEQ ID NO: 71) and MTSAIGILPV (SEQ ID NO: 80) (number 5) peptides. Irrelevant HLA A2-ALWGPDPAAA (preproinsulin) (SEQ ID NO: 86) tetramer used as an irrelevant control. (FIG. 10B) Chromium release cytotoxicity assay performed for the T-cell lines from patient 37 using autologous melanoma. The T-cell line to melanoma cell ratio displayed is based on total T-cell number. Insufficient cells were available from patient 12 to perform the killing assay. (FIG. 10C) Cytotoxicity assay as in B, but with cell numbers adjusted according to EAAGIGILTV (SEQ ID NO: 71) tetramer positivity shown in (FIG. 10A), to give 2 EAAGIGILTV tetramer⁺ cell per 3 melanoma cells, for both the EAAGIGILTV and MTSAIGILPV primed T-cell lines. P values are displayed for an unpaired one-tailed t-test.

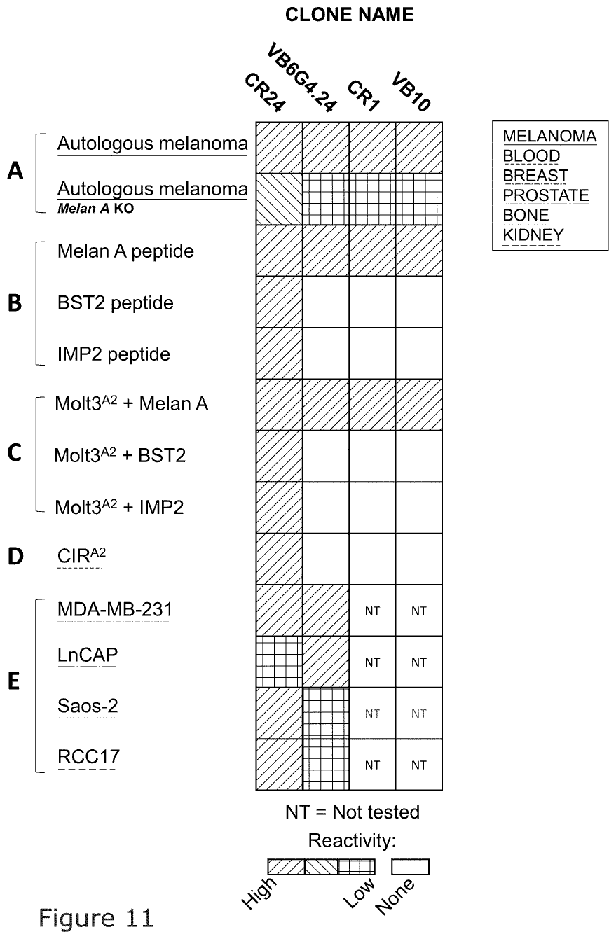

FIG. 11 shows summarised preliminary data from other potentially multipronged T-cells. T-cell clones (VB6G4.24, CR1 and VB10) also grown from TIL patient MM909.24 recognise the Melan A peptide (EAAGIGILTV) (SEQ ID NO: 71) but not BST2 (LLLGIGILVL) (SEQ ID NO: 72) or IMP2 (NLSALGIFST) (SEQ ID NO: 73) peptides (neither as exogenous peptide nor from transduced protein expressed by MOLT3s). The CDR3 sequence of the beta TCR chain from VB6G4.24 appeared in clonotyping data for all ten cancer cell lines in FIG. 2, suggesting that this clone responds to multiple cancer cells lines but not by recognition of the IMP2 or BST2 peptides.

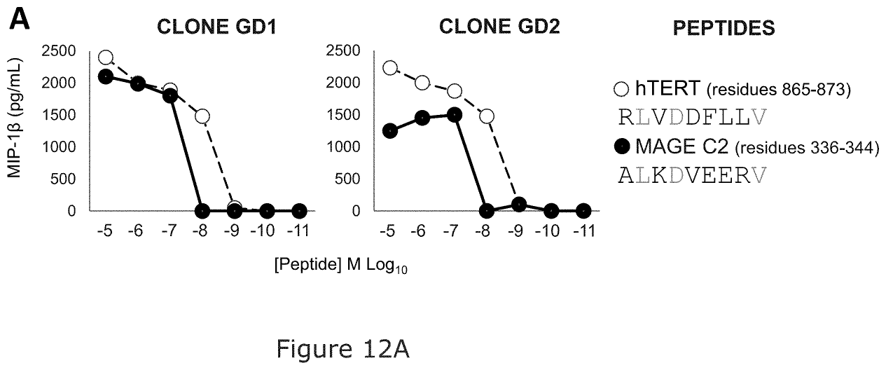

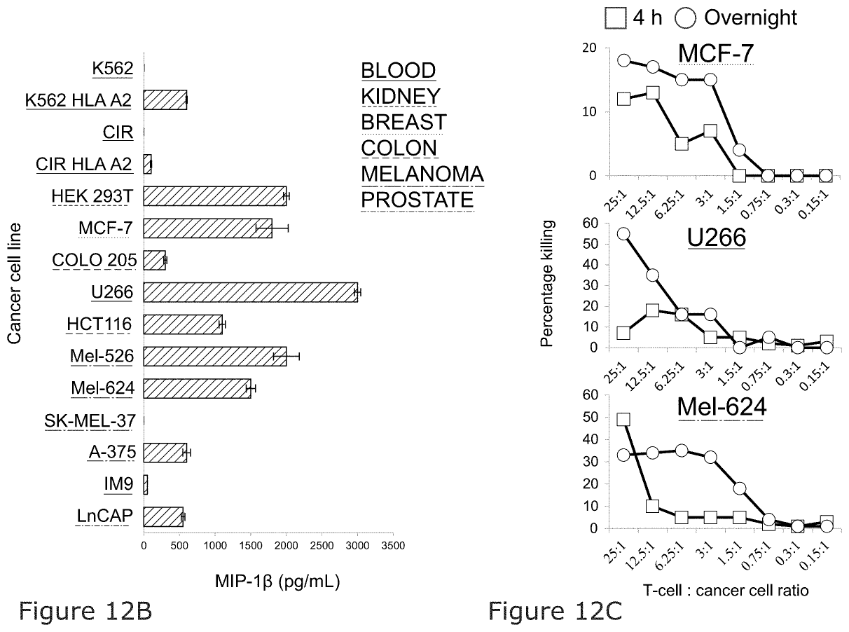

FIGS. 12A-C show the peptide cross-reactivity of other multipronged T-cells. Clones GD1 and GD2 recognise different peptides than clone CR24. (FIG. 12A) HLA A2− restricted clones GD1 and GD2 grown from different donors express different T-cell receptors but recognise the same peptides from human telomerase reverse transcriptase (hTERT) and MAGE C2, as shown. Only the grey amino acid residues are common to each of the peptides. Overnight activation assay with each of the clones using decreasing concentrations of each of the peptides. Supernatants were harvested and used for MIP-113 ELISA. (FIG. 12B) Preliminary screening of GD1 for recognition of cancer cell lines with different tissue origin. Overnight activation assay and MIP-1β ELISA. (FIG. 12C) Chromium release cytotoxicity assay with cell lines identified in (FIG. 12B) as being good targets of GD1. Percent specific lysis assessed after 4 h and overnight incubation.

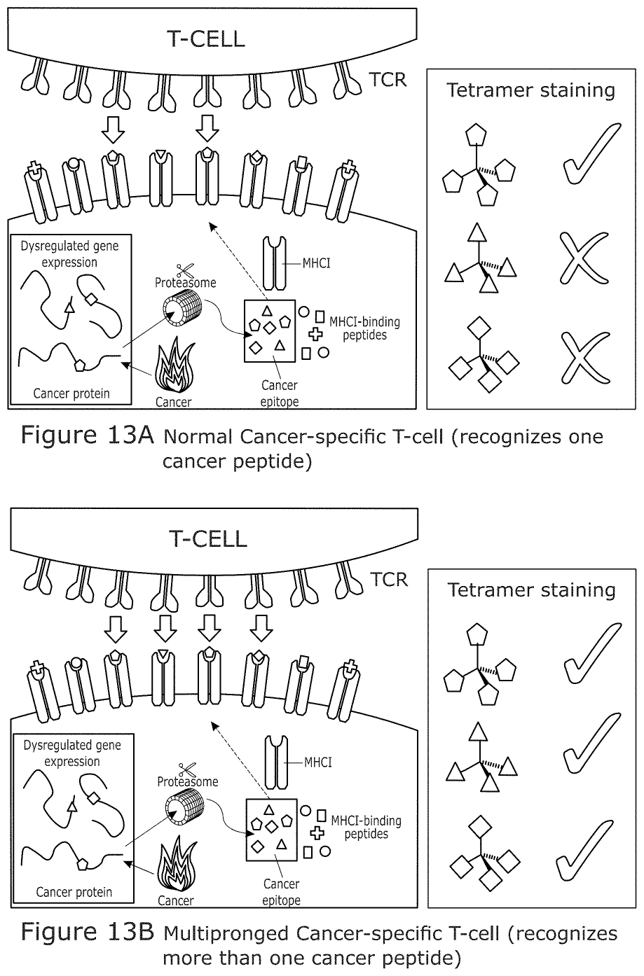

FIGS. 13A-B show multipronged cancer specific T-cells and T-cell receptors differ from normal anti-cancer T-cells. (FIG. 13A) Conventionally, anti-cancer T-cells recognise cancer cells when the TCR binds to a peptide derived from cancer antigens as shown in A. These T-cells do not respond to other cancer-derived peptides. (FIG. 13B) Unusually, multipronged anti-cancer T-cells bear TCRs that recognise multiple different cancer peptides. It is far more difficult for cancer cells and a developing tumour to escape from multipronged T-cells. Consequently, the use of multipronged TCRs is desirable in cancer immunotherapy approaches.

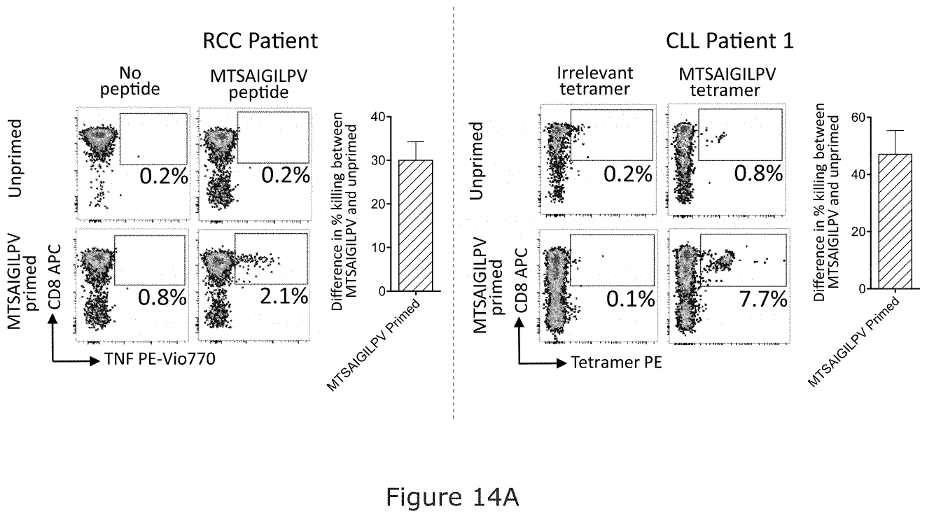

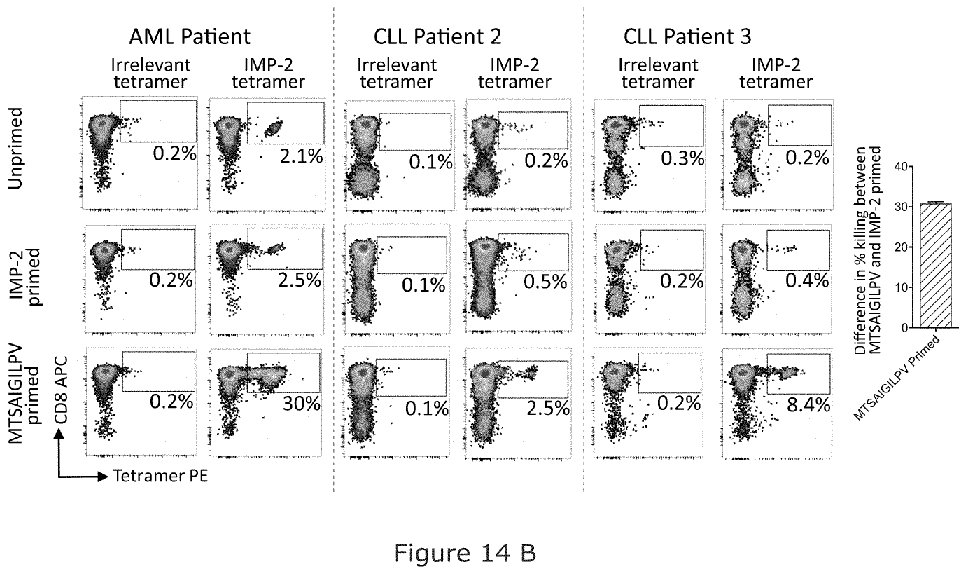

FIGS. 14A-B show super-agonist peptide MTSAIGILPV primed a greater proportion of cancer-specific T-cells leading to enhanced killing of autologous cancer. (FIG. 14A) CD8 T-cells from a renal cell carcinoma (RCC) and chronic lymphocytic leukaemia (CLL) patient were left unprimed or primed with MTSAIGILPV peptide for 28 days. A TAPI-0 assay (RCC patient) or tetramer staining (CLL patient) demonstrated the presence of MTSAIGILPV (SEQ ID NO: 80) specific T-cells. The MTSAIGILPV (SEQ ID NO: 80) primed CD8s killed more autologous cancer cells than the unprimed T-cells. (FIG. 14B) CD8 T-cells from an acute myeloid leukaemia (AML) patient and two CLL patients were left unprimed or primed with either wild-type IMP-2 (NLSALGIFST) (SEQ ID NO: 73) or MTSAIGILPV (SEQ ID NO: 80) peptide for 28 days. Analysis performed with IMP-2 tetramer revealed that the unprimed and IMP-2 primed conditions had similar proportions of IMP-2 specific T-cells, whereas MTSAIGILPV broke tolerance and induced a greater proportion of IMP-2 cells. T-cells from CLL patient 3 were used in a killing assay and the MTSAIGILPV (SEQ ID NO: 80) primed T-cells killed more CLL cells than the IMP-2 primed CD8s.

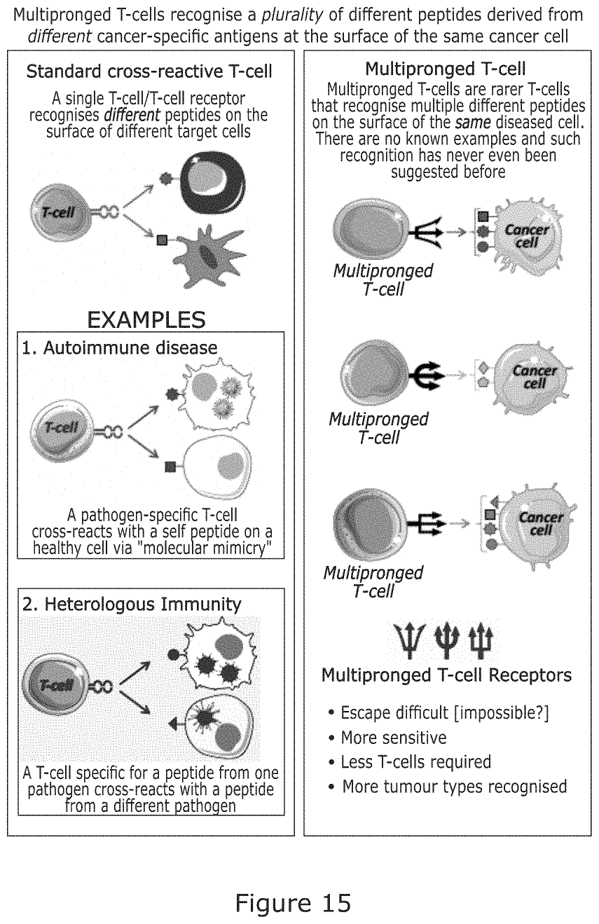

FIG. 15 shows a schematic of how the multipronged T-cells recognise a plurality of different peptides derived from the different cancer-specific antigens at the surface of the same cancer cell.

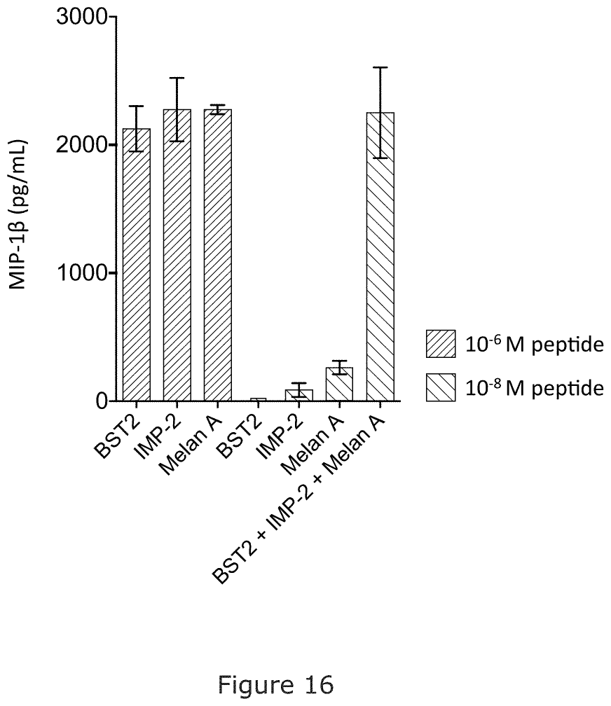

FIG. 16 shows multipronged T-cells recognise peptides additively and at low concentration. Multipronged T-cell clone CR24 recognizes peptides from BST2 (LLLGIGILVL) (SEQ ID NO: 72), Melan A (EAAGIGILTV) (SEQ ID NO: 71) and IMP2 (NLSALGIFST) (SEQ ID NO: 73). CR24 responded to all three individual peptides at 10-6 M, but responses dropped when peptides were at 10-8 M. However, CR24 exhibited good activation when each peptide was present at 10-8 M within a mix of peptides. This demonstrates how multipronged T-cells can sensitively target cancer cells by recognition of multiple peptides from different proteins expressed by the same cell.

### CLAUSES

1. A peptide, wherein the amino acid sequence of the peptide comprises an amino acid sequence selected from the group consisting of:

(SEQ ID NO: 80)
MTSAIGILPV;
(SEQ ID NO: 77)
ITSAIGILPV;
(SEQ ID NO: 76)
ITSAIGVLPV;
(SEQ ID NO: 78)
MTSAIGVLPV;
(SEQ ID NO: 79)
QTSAIGVLPV;
(SEQ ID NO: 81)
LTSAIGVLPV;
(SEQ ID NO: 82)
ITSGIGVLPV;
(SEQ ID NO: 83)
ITSAIGVLPI;
(SEQ ID NO: 84
QTSAIGILPV;
and
(SEQ ID NO: 85)
ITSAIGVLFV.

2. A peptide, wherein the amino acid sequence of the peptide consists of an amino acid sequence having at least 80% sequence identity to a sequence selected from the group consisting of:

(SEQ ID NO: 80)
MTSAIGILPV;
(SEQ ID NO: 77)
ITSAIGILPV;
(SEQ ID NO: 76)
ITSAIGVLPV;
(SEQ ID NO: 78)
MTSAIGVLPV;
(SEQ ID NO: 79)
OTSAIGVLPV;
(SEQ ID NO: 81)
LTSAIGVLPV;
(SEQ ID NO: 82)
ITSGIGVLPV;
(SEQ ID NO: 83)
ITSAIGVLPI;
(SEQ ID NO: 84)
QTSAIGILPV;
and
(SEQ ID NO: 85)
ITSAIGVLFV.

3. The peptide according to clause 2, wherein the amino acid sequence of the peptide consists of an amino acid sequence having at least 90% sequence identity to a sequence selected from the group consisting of:

(SEQ ID NO: 80)
MTSAIGILPV;
(SEQ ID NO: 77)
ITSAIGILPV;
(SEQ ID NO: 76)
ITSAIGVLPV;
(SEQ ID NO: 78)
MTSAIGVLPV;
(SEQ ID NO: 79)
QTSAIGVLPV;
(SEQ ID NO: 81)
LTSAIGVLPV;
(SEQ ID NO: 82)
ITSGIGVLPV;
(SEQ ID NO: 83)
ITSAIGVLPI;
(SEQ ID NO: 84)
QTSAIGILPV;
and
(SEQ ID NO: 85)
ITSAIGVLFV.

4. The peptide according to clause 2, wherein the amino acid sequence of the peptide consists of an amino acid sequence selected from the group consisting of:

(SEQ ID NO: 80)
MTSAIGILPV;
(SEQ ID NO: 77)
ITSAIGILPV;
(SEQ ID NO: 76)
ITSAIGVLPV;
(SEQ ID NO: 78)
MTSAIGVLPV;
(SEQ ID NO: 79)
QTSAIGVLPV;
(SEQ ID NO: 81)
LTSAIGVLPV;
(SEQ ID NO: 82)
ITSGIGVLPV;
(SEQ ID NO: 83)
ITSAIGVLPI;
(SEQ ID NO: 84)
QTSAIGILPV;
and
(SEQ ID NO: 85)
ITSAIGVLFV.

5. The peptide according to clause 2, wherein the amino acid sequence of the peptide is MTSAIGILPV (SEQ ID NO: 80) or an amino acid sequence having at least 80% sequence identity thereto.

6. The peptide according to clause 2, wherein the amino acid sequence of the peptide is ITSAIGILPV (SEQ ID NO: 77) or an amino acid sequence having at least 80% sequence identity thereto.

7. The peptide according to clause 5, wherein the amino acid sequence of the peptide is MTSAIGILPV (SEQ ID NO: 80).

8. The peptide according to clause 6, wherein the amino acid sequence of the peptide is ITSAIGILPV (SEQ ID NO: 77).

9. A fusion polypeptide comprising (a) two or more peptides according to any one of clauses 1 to 8 or (b) one or more peptides according to any one of clauses 1 to 8 and another immunogenic peptide.

10. The peptide according to any one of clauses 1 to 8 or fusion polypeptide according to any clause 9 wherein said peptide is presented by a human leukocyte antigen (HLA) class I molecule is selected from the group comprising: HLA A, HLA A2 or HLA 24 or HLA A1 or HLA A3.

11. The peptide or fusion polypeptide according to clause 10 wherein said molecule is HLA A2.

12. The peptide or fusion polypeptide according to any one of clauses 1 to 11 which, when administered to a subject, primes the production of anti-cancer T-cells that act as effector T-cells and/or T-cells expressing said TCR that recognises a plurality of cancer antigens when said antigens are presented by the same cancer cell at a cell surface by human leukocyte antigen (HLA) class I molecule and wherein said antigens are distinct from each other and are presented by cells from different types of cancer.

13. A polynucleotide encoding the peptide or fusion polypeptide according to any one of clauses 1 to 12.

14.A vector comprising the polynucleotide according to clause 13.

15. The vector according to clause 14 which is chimeric polynucleotide comprising an open reading frame encoding the peptide or fusion polypeptide and a heterologous promoter and/or other transcription control element such as a terminating signal operably linked thereto.

16. A vaccine comprising said peptide or fusion polypeptide or polynucleotide or vector according to any one of clauses 1 to 15.

17. A pharmaceutical composition comprising said peptide or fusion polypeptide or polynucleotide or vector according to any one of clauses 1 to 15.

18. An immunogenic composition comprising said peptide or fusion polypeptide or polynucleotide or vector according to any one of clauses 1 to 15.

19. A combination therapeutic for the treatment of cancer comprising: the peptide, fusion polypeptide polynucleotide, vector, vaccine or pharmaceutical composition or immunogenic composition according to any one of clauses 1 to 18 in combination with a further cancer therapeutic agent.

20. The peptide, polynucleotide, vector, vaccine or pharmaceutical composition or immunogenic composition or combination therapeutic according to any one of clauses 1 to 18 for use in treating cancer.

21. Use of the peptide, fusion polypeptide, polynucleotide, vector, vaccine or pharmaceutical composition or immunogenic composition or combination therapeutic according to any one of clauses 1 to 19 in the manufacture of a medicament for treating cancer.

22. A method of treating a subject having, or suspected of having cancer, comprising administering the peptide, fusion polypeptide, polynucleotide, vector, vaccine or pharmaceutical composition or immunogenic composition or combination therapeutic according to any one of clauses 1 to 19 to said subject.

23. The peptide, fusion polypeptide, polynucleotide, vector, vaccine or pharmaceutical composition or immunogenic composition or combination therapeutic or the peptide, polynucleotide, vector, vaccine or pharmaceutical composition or immunogenic composition or combination therapeutic for use, or the use or method according to any one of clauses 1 to 22 wherein said cancer is selected from the group consisting of: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, blood, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumour, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

24. The peptide, fusion polypeptide, polynucleotide, vector, vaccine or pharmaceutical composition or immunogenic composition or combination therapeutic or the peptide, polynucleotide, vector, vaccine or pharmaceutical composition or immunogenic composition or combination therapeutic for use, or the use or method according to clause 23 wherein said cancer is skin cancer e.g., melanoma, renal cell carcinoma or leukaemia.

25. An antigen pool comprising of two or more peptides, said peptides having an amino acid sequence selected from:
(a) the amino acid sequence consisting of MTSAIGILPV (SEQ ID NO: 80) or an amino acid sequence having at least 80% sequence identity thereto, or an amino acid sequence comprising the sequence of MTSAIGILPV (SEQ ID NO: 80);
(b) the amino acid sequence consisting of ITSAIGILPV (SEQ ID NO: 77) or an amino acid sequence having at least 80% sequence identity thereto, or an amino acid sequence comprising the sequence of ITSAIGILPV (SEQ ID NO: 77);
(c) the amino acid sequence consisting of ITSAIGVLPV (SEQ ID NO: 76) or an amino acid sequence having at least 80% sequence identity thereto, or an amino acid sequence comprising the sequence of ITSAIGVLPV (SEQ ID NO: 76);
(e) the amino acid sequence consisting of MTSAIGVLPV (SEQ ID NO: 78) or an amino acid sequence having at least 80% sequence identity thereto, or an amino acid sequence comprising the sequence of MTSAIGVLPV (SEQ ID NO: 78);
(e) the amino acid sequence consisting of QTSAIGVLPV (SEQ ID NO: 79) or an amino acid sequence having at least 80% sequence identity thereto, or an amino acid sequence comprising the sequence of QTSAIGVLPV (SEQ ID NO: 79);
(f) the amino acid sequence consisting of LTSAIGVLPV (SEQ ID NO: 81) or an amino acid sequence having at least 80% sequence identity thereto, or an amino acid sequence comprising the sequence of LTSAIGVLPV (SEQ ID NO: 81);
(g) the amino acid sequence consisting of ITSGIGVLPV (SEQ ID NO: 82) or an amino acid sequence having at least 80% sequence identity thereto, or an amino acid sequence comprising the sequence of ITSGIGVLPV (SEQ ID NO: 82);
(h) the amino acid sequence consisting of ITSAIGVLPI (SEQ ID NO: 83) or an amino acid sequence having at least 80% sequence identity thereto, or an amino acid sequence comprising the sequence of ITSAIGVLPI (SEQ ID NO: 83);
(i) the amino acid sequence consisting of QTSAIGILPV (SEQ ID NO: 84) or an amino acid sequence having at least 80% sequence identity thereto, or an amino acid sequence comprising the sequence of QTSAIGILPV (SEQ ID NO: 84); and
(j) the amino acid sequence consisting of ITSAIGVLFV (SEQ ID NO: 85) or an amino acid sequence having at least 80% sequence identity thereto, or an amino acid sequence comprising the sequence of ITSAIGVLFV (SEQ ID NO: 85).

26. The antigen pool according to clause 25 comprising (i) a peptide having an amino acid sequence consisting of the amino acid sequence MTSAIGILPV (SEQ ID NO: 80) or an amino acid sequence having at least 80% sequence identity thereto, or comprising the amino acid sequence TSAIGILPV (SEQ ID NO: 80) and/or (ii) a peptide having the amino acid sequence consisting of the amino acid sequence ITSAIGILPV (SEQ ID NO: 77) or an amino acid sequence having at least 80% sequence identity thereto, or comprising the amino acid sequence ITSAIGILPV (SEQ ID NO: 77).

27. Use of one or more peptides or fusion polypeptide according to any one of clauses 1 to 11 or fusion polypeptides according to clause 12 as antigens in the ex vivo stimulation and/or amplification of T-cells derived from a human suffering from cancer, in particular, for subsequent reintroduction of said stimulated and/or amplified T-cells into the said human for the treatment of the said cancer in the said human.

28. Use of the antigen pool according to clause 25 or clause 26 in the ex vivo stimulation and/or amplification of T-cells derived from a human suffering from cancer, in particular, for subsequent reintroduction of said stimulated and/or amplified T-cells into the said human for the treatment of the said cancer in the said human.

29. An ex vivo method of stimulating and/or amplifying T-cells which comprises contacting said T-cells, optionally together with antigen-presenting cells, with one or more peptides according to any one of clauses 1 to 11 or one or more fusion polypeptides according to clause 12 as antigens or with the antigen pool of clauses 25 or clause 26.

30. A population of stimulated and/or amplified T-cells obtainable or obtained according to the method of clause 29.

31. A method of treatment of cancer in a human which comprises taking from said human T-cells optionally together with antigen-presenting cells, stimulating and/or amplifying said T-cells in the presence of one or more peptides according to any one of clauses 1 to 11 or one or more fusion polypeptides according to clause 12 as antigens, or in the presence of the antigen pool of clauses 25 or clause 26, and reintroducing some or all of said stimulated and/or amplified T-cells into the human.

32. An antigen-presenting cell which presents a peptide according to any one of clauses 1 to 8 in complex with an HLA peptide.

### Methods and Materials

### General Cell Culture Reagents and Cell Lines

RMPI-1640 with 2 mM L-glutamine, 100 U/mL penicillin and 100 μg/mL streptomycin (termed R0) was supplemented with either 5% (R5) or 10% (R10) foetal calf serum. T-cell media was R10 with added 10 mM HEPES buffer, 0.5× non-essential amino acids, 1 mM sodium pyruvate, 20-200 IU/mL of IL-2 (Aldesleukin, Proleukin, Prometheus, San Diego, Calif., USA) and 25 ng/mL of IL-15 (Peprotech, Rocky Hill, N.J., USA). D10-F12 media was made as for R10 using DMEM-F12. Unless otherwise stated tissue culture reagents were from Life Technologies (Carlsband, Calif., USA). Cell lines C1R, T2 and IM9 were cultured as suspension cells in R10. Malignant melanoma cell lines Mel-526, Mel-624, FM-2, FM-56, SK-MEL-37 and A-375 were cultured as adherent cells in R10. Melanoma MM909.24 and renal cell carcinoma RCC17 were obtained from patients treated at the CCIT and cultured as suspension cells in R10 and D10-F12 respectively. Other cancer cell lines were maintained as described by the ATCC; breast adenocarcinoma MDA-MB- 231 (ATCC® HTB-26™) and MCF-7 (ATCC® HTB-22™); prostate adenocarcinoma LnCAP (ATCC® CRL-1740™); colorectal carcinomas COLO 205 (ATCC® CCL-222™) and HCT116 (ATCC® CCL-247™); lung carcinoma H69 (ATCC® HTB-119™); liver hepatocellular carcinoma HepG2 (ATCC® HB-8065™); cervical carcinoma MS751 (ATCC® HTB-34™); acute lymphoblastic leukaemia MOLT3 (ATCC® CRL-1552™); chronic myeloid leukaemia K562 (ATCC® CRL-3344™); myeloma/plasmacytoma U266 (ATCC® TIB-196™) osteosarcomas U-2 OS (ATCC® HTB-96™) Saos-2 (ATCC® HTB-85™) and TK143 (ATCC® CRL-8303™); HEK293T embryonic kidney cell (ATCC® CRL-1573™); acute monocytic leukaemia THP-1 (ATCC® TIB-202™); and kidney carcinoma A-498 (ATCC® HTB-44™).

### Melanoma Tumour Infiltrating Lymphocytes Recognise Multiple Cancer Cell Types

Stage IV metastatic melanoma patient MM909.24 underwent rapid tumour infiltrating therapy at the Centre for Cancer Immunotherapy (CCIT), Herlev Hospital, Copenhagen [1]. To date, this patient has experienced lasting remission. Chromium release cytotoxicity assay was used to assess reactivity towards cancer cell lines: autologous melanoma (MM909.24), MDA-MB-231, MCF-7, LnCAP and RCC17. Cell lines (1×10⁶ cells) were labelled for 1 h with 30 μCi of sodium chromate (51Cr) (Perkin Elmer, Waltham, Mass., USA), leached for 1 h, then cultured with TILs overnight. A 10:1 TIL to target cell (2000 cells per well) ratio was used. After overnight incubation supernatants were harvested, mixed with scintillant and read using a microbeta counter and specific lysis calculated [2]. Further cancer cell lines were tested using a TNF processing inhibitor-0 (TAPI-0) assay [3]; TILs were harvested from culture washed with RO and rested overnight in R5 media. On the day of the activation assay, cells were harvested then counted and 100,000 incubated with 30 μM TAPI-0 (Sigma-Aldrich) anti-TNF-PE-Vio770TM (clone cA2, Miltenyi Biotech) and anti-CD107a-PE (clone H4A3, BD Biosciences) antibodies in wells of a 96 U well plate. Cancer cell lines were added to give a TIL to target cell ratio of 1:2. In addition to the cancer cell lines above the following were also used; COLO 205, H69, HepG2, MS751 and Saos-2. The cells were incubated for 4-5 h at 37° C. then stained at RT for 5 min with 2 μL of LIVE/DEAD fixable dead cell stain ViVid (Life Technologies) that had been diluted 1:40 using PBS. Antibodies to detect surface markers were added directly to each sample without washing; anti-CD8-APC (clone BW135/80, Miltenyi Biotech) and anti-CD3-peridinin chlorophyll (PerCP) (clone BW264/56, Miltenyi Biotech). Data was acquired on a BD FACS Canto II (BD Biosciences) and analysed with FlowJo software (TreeStar Inc., Ashland, Oreg., USA). Activated TILs (CD107a+ and/or TNF+) were sorted on a BD FACS Aria (BD Biosciences, San Jose, Calif., USA) and used for next generation sequencing of the T-cell receptor (TCR) chains as previously described [4].

### The Strategy for Identifying Peptides Recognised by Orphan CD8 Clones

T-cell clones of unknown peptide specificity (termed orphan clones) were generated by culturing 0.5 cells/well in of 96 U well plates in T-cell media with 50,000 irradiated (3000-3100 cGy) allogenic peripheral blood mononuclear cells (PBMCs) from three donors and 1-2 μg/mL of phytohaemagglutinin (PHA). PBMCs were separated from blood by standard density gradient centrifugation. If needed, red blood cells were lysed using ammonium chloride solution. Blood was procured as buffy coats' from the Welsh Blood Service (Pontyclun, Wales, UK). All human tissue was obtained and handled in accordance with Cardiff University's guidelines to comply with the UK Human Tissue Act 2004. T-cell clones were screened against autologous melanoma (MM909.24) and in some case cancer cell lines of different tissue origin. Clones of interest were grown to large number in T25 flasks using the PBMC and PHA method as above. Combinatorial peptide library (CPL) and cancer antigen database screening was performed to find peptides recognized by orphan clones. Combinatorial peptide libraries were synthesized and used as previously described [5,6]. Briefly, long-term storage was at −80° C. as 20 mM DMSO stocks with 1 mM working dilutions made in sealable (silicone sealing mat, AxyGen® AxyMat™, Corning, N.Y., US) 2 mL deep round-well plates (AxyGen®, Corning) with R0 (as for R10 but with no serum), which were stored at 4° C., then vortexed (MixMate®, Eppendorf®, Hamburg, Germany) at 1300 rpm for 1 min, then centrifuged (400g, 5 mins) before use. Each sub-library was used at a concentration of 100 μM with respect to total peptide concentration. The CPL data was run via a database, which contains the amino acid sequences of proteins expressed by cancers (manuscript in preparation). The cancer antigen database will be available online as part of the PI CPL (peptide identification combinatorial peptide library) webtool hosted by Warwick University's Systems Biology Centre (http://wsbc.warwick.ac.uk/wsbcToolsWebpage/user_cases.php). Candidate peptides from the database were automatically ranked based on their likelihood of being recognised by a clone, with the top 20 being tested in peptide titration assays.

### CR24 Recognises Multiple Cancer Cell Types

HLA A2+ Melanomas, MM909.24 (autologous), Mel-526, Mel-624, and HLA A2+ non-melanomas, CIR-HLA A2, MDA-MB-231, Saos-2, U205, A498, TK143, HEK293T, COLO 205, HCT116, HeLa, HepG2 and THP1 were used as target cells in a TAPI-0 assay, which is described above. HLA A2neg melanomas FM-2 and FM-56, and wild-type C1Rs (HLA A2neg) were used as controls.

### Combinatorial Peptide Library (CPL) and Cancer Antigen Database Screening of Clone CR24

CR24 was rested overnight in R0 then 30,000 used per well of the decamer CPL screen (details above). The peptide length preference of CR24 had previously been established using sizing scan assays [7] (data not shown). T2 cells (60,000 per well) were used as antigen presenting cells. The assay was performed in R5 and supernatants harvested for MIP-1β enzyme linked immunosorbent assay (ELISA) according to the manufacturer's instructions (R&D Systems, Minneapolis, Minn., USA).

### CR24 Recognises Three HLA A2 Restricted Peptides from Different Cancer Proteins

CR24 was cultured overnight in R5, then 30,000 used per well of a 96 U well plate with decreasing concentrations of peptides. After overnight incubation supernatants were used MIP-1 β ELISA according to the manufacturer's instructions (R&D Systems, Minneapolis, Minn., USA). For tetramer analysis CR24 (20,000-50,000 per sample) was stained in 5 mL polypropylene tubes suitable for flow cytometry. Cells were treated in 100 μL of FACS buffer (PBS+2% FBS) with 50 nM Dasatinib (a protein kinase inhibitor) for 30 min at 37° C. and phycoerythrin (PE) conjugated tetramer (0.5 μg) added directly to the sample before being moved to ice for a further 30 min [8]. Tetramer was washed with 3 mL of FACS buffer (700 g, 5 min) then labelled with 0.5 μg (10 μg/mL) of mouse anti-PE unconjugated antibody (clone PE001, BioLegend, London, UK) for a further 20 min on ice [8]. To test if CR24 could recognise endogenously express antigen MOLT3 cells were used to express various proteins. Codon optimised full-length human HLA A2 (IMGT/HLA Acc No: HLA00005), MLANA (Melan A) (UniProtKB Q16655), BST2 (UniProtKB Q10589), IGF2BP2 (IMP2) (UniProtKB Q9Y6M1), COL6A2 (α2 subunit of collagen type VI) (UniProtKB P12110) and Zika virus (Rio-U1) ancC (GenBank KU926309.2) genes were synthesized (Genewiz, South Plainfield, N.J., USA) and cloned into the 3rd generation lentiviral transfer vector pELNS (kindly provided by Dr. James Riley, University of Pennsylvania, Pa., USA). The pELNS vector contains a rat CD2 (rCD2) marker gene separated from the gene of interest by a self-cleaving 2A sequence. Lentiviral particle production, calcium chloride transfection and rCD2-based purification of cells were performed as previously described [9].

### Clone CR24 is Able to Recognise Autologous Melanoma Lacking Melan A Expression

To demonstrate that CR24 can target autologous melanoma through multiple antigens, guide RNAs to ablate Melan A expression using CRISPR/Cas9 were designed using the cripsr.mit.edu webtool, applied and the Melan A gene sequenced to confirm disruption (data not shown). Intracellular staining for Melan A was performed using Cytofix/Cytoperm™ reagents according to manufacturer's instructions (BD Biosciences). A primary unconjugated rabbit anti-Melan A antibody (clone EP1422Y) (Abcam, Cambridge, UK) was used with a secondary PE conjugated goat anti-rabbit antibody. Wild type and Melan A KO MM909.24 melanomas were used TAPI-0 assays, as described above, with both TILs and CR24.

### T-Cells that Recognise the Same Three Peptides as CR24 are Present in Healthy HLA A2+ Donors

To generate T-cell peptide lines, CD8 T-cells were purified from the PBMCs of HLA A2+ donors using CD8 microbeads according to the manufacturer's instructions (Miltenyi Biotech, Bergisch Gladbach, Germany). Purified CD8 cells (3×10⁶) were co-incubated with autologous CD8neg cells (6-8×10⁶) in 24 well plates in 2 mL of T-cell media, but with no IL-15. 25 μM of each peptide was used. The cultures had 50% of the media changed thrice weekly. Tetramer staining was performed as above, using 500,000 cells per tube. Each T-cell line was used in an IFNγ enzyme linked immunosorbent spot (ELISpot) assay with cell lines MDA-MB-231, melanoma MM909.24 and Saos-2. 50,000 T-cells and 15,000 cancer cells were used per well. Incubation was performed for 48 h, and the assay developed according to the manufacturer's instructions (Mabtech, Nacka Strand, Sweden).

### Super-Agonist Peptides Prime Multi-Pronged T-Cells for Improved Cancer Cell Recognition

CPL assay of CR24 was performed as described above. Candidate peptide agonists were designed using the CR24 CPL and an online algorithm (http://wsbc.warwick.ac.uk/wsbcToolsWebpage/user_cases.php). Priming of CD8 T-cells from healthy donors, tetramer staining, and chromium release cytotoxicity assays were performed as described above.

### Other Melan A clones do not Recognise the BST2 and IMP2 Peptides Seen by CR24

TAPI-0 and activation assays (ELISA) were performed for VB6G4.24, CR1 and VB10, as described above for CR24. The data was summarised in tabular from.

### Clone Recognition of Peptides from Cancer Antigens HTERT and MAGE C2

Clones GD1 and GD2 were grown from the peripheral blood of different HLA A2+ healthy donors. The clones were used in overnight activation assays with decreasing concentrations of respective peptides, and supernatants used for MIP-1β ELISA, as described above. An overnight activation was performed with GD1 and target cells; K562, K562 HLA A2, CIR, CIR HLA A2, HEK 293T, MCF-7, COLO 205, U266, HCT116, Mel-526, Mel-624, SK-MEL-37, A375, IM9 and LnCAP. Supernatants were harvested and used for MIP-1β ELISA. A chromium release cytotoxicity assay was performed, as above, with cell lines MCF-7, U266 and Mel-624. Incubation times of 4 h and overnight, with varying T-cell to target cell ratios were used.

### Ex Vivo Stimulation of T-Cells Using Peptides or Fusion Polypeptides

T-cells from a healthy donor, or patient with a given cancer, can be stimulated outside of the body (ex vivo) to activate T-cell clones that recognise specified peptides or fusion polypeptides of the invention, and subsequently rapidly expanded to generate large numbers of reactive T-cells, where resultant anti-tumour activity might be anticipated. A number of steps are involved to employ this method.

### A) Isolation of Relevant Patient Immune Cells

T-cells from the donor (healthy or cancer patient) must be isolated but also autologous antigen presenting cells (APCs) may be required. The source of the immune cells can be obtained from peripheral blood through a blood draw or apheresis. Alternatively, T-cells can be isolated from the tumour infiltrating lymphocytes (TILs) obtained from fresh biopsy or resection of a patient's tumour. APCs may be cluster of differentiation (CD)14-positive monocytes or alternatively dendritic cells (DCs) which would be derived from the monocyte fraction of the apheresis product. DCs can be generated by methods such as positive isolation via CD14 capture (for example, anti-CD14 antibodies conjugated to magnetic beads, where CD14-positive cells are labelled with the beads and captured on a magnetic column) or isolation via their adhesive properties, for example, adherence to tissue culture plastics by incubation of peripheral blood mononuclear cells (PBMCs) with cell culture dishes for a period of 4-48 hr to allow adherence of monocytes. DCs can be generated from the CD14-positive or adherent immune cell fractions by well-described methods utilising cytokines such as, but not limited to: GM-CSF, IL-4, TNFα, IL-6, Prostaglandin E2. Incubation with such cytokines over the course of 2-7 days allows differentiation of the CD14+ monocytes into DCs, typically that will have lost the expression of CD14 and upregulated expression of DC markers such as CD11c, high levels of MHC Class II, etc. The nature of the T-cells for selection and/or stimulation could be the monocyte-depleted fraction of PBMC (in the case of apheresis origin of T-cells), pan-T cell isolation using isolation techniques based on the expression of markers such as CD3, or presence or absence of markers of specific T cell subsets, for example but not limited to, CD4, CD8, CD45RO, CD45RA, CCR7, CD62L, CD27 etc.

### B) Selection of T-Cells Specific for Peptides of the Invention

Methods can be employed to select T-cells prior to stimulation with APCs. Such methods would include peptide-HLA (pHLA) multimer approaches such as tetramer, pentamer, dextramer or similar, to label T-cells that express TCRs that recognise the given pHLA (e.g. HLA-A*02:01). Such pHLAs would be defined based on the predicted propensity of the peptides of the invention to bind specific HLA allotypes (based on prediction algorithms). The multimer could possess a tag, such as phycoerythrin (PE) which could be isolated using fluorescent activated sorting or via an anti-PE antibody conjugated to magnetic beads. Alternatively, an antibody to the tag could be directly conjugated to magnetic beads.

### C) Stimulation of T-Cells

In order to potentiate pre-existing (memory) or stimulate new (naïve) T-cell responses from cancer patients to peptides of the invention, the patient's T-cells can be exposed to APCs that are presenting said peptides on the surface in the context of Class I HLA complexes. For example, peptides or fusion polypeptides of the invention (anticipated to be expressed by a patient's tumour) could be exogenously delivered to a patient's APCs to result in said peptides being presented on HLA complexes at the surface. Introduction of these peptides could be through delivery of exogenous synthetic peptides or pools of peptides, fusion polypeptides or mRNA-based methods of delivery of a fusion polypeptide.

Methods of stabilized, mature, mRNA delivery to the APC (that is, transfection) could include classical reagents such as polyethylenimine (PEI) or calcium phosphate for nucleic acid delivery into cells. Alternatively, efficient transfection can be achieved using lipid-based reagents for transfection into APCs. Delivery of fusion polypeptides of the invention to APCs with such methods described should result in the expression of polypeptides in the cytoplasm of the APC, which in turn will result in cellular processing of peptide fragments from the polypeptides for presentation on Class I and Class II HLA molecules. When T-cells (either selected as described in (b) or unselected T-cells from apheresis or TIL sources) are co-cultured with APCs expressing peptide-HLA complexes at the cell surface, those T-cells possessing TCRs that have specificity for a given pHLA will be stimulated by engaging with the pHLA complex in addition to co-stimulatory molecules and signals from the APC. This will result in activation, differentiation and proliferation of the engaged T-cell.

### D) Enrichment of Stimulated T-Cells

T-cells that have been stimulated by APCs that are expressing and/or presenting peptides of the invention can be further enriched prior to an expansion step if required. Markers of T-cell activation (such as CD137, CD107a, CD69, OX40 or other surface marker associated with an activated state) or T-cell functional responses (for example, T-cells secreting cytokines such as TNFα or IFNγ) could be selected for, to enrich the T-cell population for those cells that might be specific for the peptides of the invention. Such enrichment methods could include cell sorting by FACS or bead-based methods of capture, for example, using antibodies to CD137 or similar that are conjugated to magnetic beads. Multiple enrichment strategies could be employed, either in parallel (for example, cells double positive for CD137 and CD69) or sequentially (for example, selecting cells positive for CD137 and subsequently selecting CD137+ cells positive for CD69). Such a positive selection should remove those T-cells that are likely not stimulated by the APCs presenting the peptides of interest.

### E) Rapid Expansion of Stimulated T-Cells

Following stimulation of T-cells with APCs that are expressing and/or presenting peptides of the invention, bulk or enriched (see (D) above) T-cells can be rapidly expanded to achieve numbers>108 total cells, using methods based on those described in the literature, with potential modifications for optimisation (for example, Jin et al., J Immunother, 2012). Such methods utilise cytokines such as IL-2 and stimulatory antibodies such as anti-CD3 as well as potential irradiated autologous cells from PBMC (termed “feeder” cells). Alternatively, stimulatory antibodies to CD3 and CD28 can be used to avoid the use of feeder cells. The process can be further automated or enhanced using specialized gas-permeable flasks (for example G-Rex flasks) or closed expansion system (for example WAVE bioreactor). Significant expansion of T-cells (100-1000 fold) can be achieved in as little as 7-14 days, depending on the numbers of T-cells at the start.

### F) Testing of Expanded T-Cells for Evidence of Immunogenicity

To demonstrate that the ex vivo autologous stimulation process has expanded T-cells that recognize target cells presenting peptides of the invention (including tumor cells), multimers corresponding to specific peptide-HLA (pHLA) complexes could be used to detect the presence of T-cells with reactivity for a particular pHLA.

Functional assays would also demonstrate the ability of the ex vivo-immunized T-cells to respond to target cells presenting peptides of the invention. This could be achieved through a variety of approaches. Firstly, cytokine release assays could be performed to test for T-cell activation from co-cultivation of the ex vivo stimulated T-cells with the target cells (for example, IFNγ ELISpot assays). Alternatively, T-cell mediated killing of target cells could be measured with cytotoxicity assays such as FACS-based methods to assess cell death of target cells (e.g., by 7-AAD measurement) co-cultured with the T-cells, or other methods such as those that monitor markers of apoptosis of target cells or measure impedance (electrical measure of cell viability) of adherent target cells plated onto specialized surfaces.

A variety of methods could be used to create target cells for such assays. For example, appropriate human cells with HLAs that match APCs used in the ex vivo stimulation could be pulsed with peptides of the invention. Further, tumor cell lines matching the HLA type of the APCs could also be assessed. Finally, primary tumor cells (in particular tumor cells from the same patient donor from which the starting T-cells and APCs used for the process were derived) could be assessed.

In conclusion, these methods can be used to demonstrate that a) human T-cells are able to be “immunized” with peptides or fusion polypeptide of the invention using autologous APCs ex vivo, b) immunized T-cells are able to be potentially enriched over non-immunized T-cells, c) immunized T-cells can be rapidly expanded to produce several log-fold higher number of total cells, and d) rapidly expanded, immunized T-cells retain the capacity to recognize target cells that express the same HLAs and peptides they were immunized against. These data would support the likelihood that an ex vivo stimulation protocol applied to cancer patients would have therapeutic value in controlling cancer.

### Results

1. Tumour infiltrating lymphocytes (TILs) derived from a metastatic melanoma patient that underwent successful immunotherapy are capable of killing and recognising autologous melanoma and HLA A2+ cancer cell lines originating from a range of cancers: breast, colon, lung, liver, prostate, cervix, bone and kidney (FIG. 1).

2. T-cell receptor clonotyping of cancer reactive TILs revealed that the same T-cells recognised multiple HLA A2+ cancer cell lines (FIG. 2). 50% of the T-cells (TCRs) recognised more than 4 cancer cell lines and, 8.6% (5 TCRs) recognised all 10 cell lines tested. Further experiments aimed at understanding the pan cancer cell line recognition resulted in the discovery that a single T-cell can recognise multiple peptides originating from different cancer proteins.

3. In order to map the peptide specificities of the T-cells from the TILs, the T-cells were firstly cloned, then screened for reactivity towards various cancer cell lines. Clone CR24 exhibited reactivity towards autologous melanoma and cancer cell lines from breast, bone, kidney, blood, colon, cervix and liver (FIG. 4). This reactivity was mediated through HLA A2 as HLAA2neg melanomas and wildtype CIR cells (HLA A2neg) were not recognised.

4. Combinatorial peptide library and cancer antigen database screening (as described in FIG. 3) of CR24 (FIG. 5) revealed multiple peptides that were predicted to be seen by CR24 (data not shown), with three of them being recognised when tested as exogenous peptide (FIG. 6). CR24 also stained with HLA A2 tetramers containing the three peptides (FIG. 6). The peptides; EAAGIGILTV (SEQ ID NO: 71) from Melan A (residues 26-35), LLLGIGILVL (SEQ ID NO: 72) from BST2 (residues 22-31) and NLSALGIFST (SEQ ID NO: 73) from IMP2 (residues 367-376). These data demonstrate that CR24 is cross-reactive for distinct peptides derived from different cancer proteins.

5. The peptides recognised by CR24 are processed and presented from endogenously expressed proteins, as CR24 was capable of recognising antigen presenting cells (MOLT3) made to stably express either Melan A, BST2 or IMP2 (FIG. 6).

6. It would be extremely difficult for cancer cells to escape from T-cells that were targeting them through more than one different cancer antigen as escape would require simultaneous mutation of all targets that lowered or ablated presentation of all cognate peptides. To demonstrate this, we targeted autologous melanoma (MM909.24) for ablation of the Melan A gene, which was confirmed by antibody staining to lack Melan A protein expression (Melan A knockout (KO)) (FIG. 7). Both the TIL from patient MM909.24 and clone CR24 recognised the Melan A knockout melanomas (FIG. 7). For CR24, reactivity against wild type autologous tumour was 71% and for the Melan A KO 55%. It is highly likely that CR24 was recognising the Melan A KO melanoma through the BST2 and/or IMP2 peptides and therefore able to mediate destruction of the melanoma.

7. CD8 T-cells able to recognise the Melan A, BST2 and IMP2 peptides seen by CR24 can be generated from the peripheral blood of healthy HLA A2+ donors (FIG. 8).

8. Super-agonists designed for multi-pronged T-cells primed a greater proportion of CD8 T-cells capable of recognising WT Melan A (EAAGIGILTV) (SEQ ID NO: 71), BST2 (LLLGIGILVL) (SEQ ID NO: 72) and IMP2 (NLSALGIFST) (SEQ ID NO: 73) peptides, compared to parallel priming with the WT peptides. Super-agonist MTSAIGVLVP (SEQ ID NO; 80) (peptide 5) seemed to be the most effective of the candidate super-agonists at priming (FIG. 9B), eliciting Melan A, BST2 and IMP2 reactive T-cells in all donors tested (n=3). Additionally, MTSAIGILPV (SEQ ID NO; 80) and ITSAIGILPV (SEQ ID NO; 77) were superior at priming Melan A (EAAGIGILTV) T-cells from metastatic melanoma patients compared to the WT EAAGIGILTV peptide (FIG. 10A), and MTSAIGILPV (SEQ ID NO; 80) also in renal cell carcinoma (RCC) and chronic lymphocytic leukaemia (CLL) patients (FIG. 14A) and acute myeloid leukaemia (AML) patients (FIG. 14B). Importantly, the MTSAIGILPV (SEQ ID NO; 80) super-agonist peptide primed T-cells exhibited superior lysis of autologous melanoma cells than the WT Melan A peptide primed T-cells (FIGS. 10B and 10 C).

9. Clones (GD1 and GD2) grown from the peripheral blood of two healthy HLA A2+ donors cross-react with different peptides than those recognised by CR24. These peptides are derived from different proteins to those recognised by the CR24 T-cell clone; RLVDDFLLV (SEQ ID NO: 74) from human telomerase reverse transcriptase (hTERT) (residues 855-873) and ALKDVEERV (SEQ ID NO: 75) from melanoma associated antigen C2 (MAGE C2) (residues 336-344). GD1 killed breast, blood and melanoma cancer cell lines (FIG. 9).

### Conclusion

The current consensus view is that cancer-specific T-cells recognise cancer cells via a single peptide antigen presented as a peptide at the cell surface in association with HLA (FIG. 10A). We have discovered that some, rare T-cells are able to recognise cancer cells through multiple peptide epitopes that differ in sequence by two or more amino acids and are derived from different cancer antigens (FIG. 10B). Cancer escape from this type of multipronged T-cell is likely to be extremely difficult.

### REFERENCES

[1] Andersen R, Donia M, Ellebaek E, Borch T H, Kongsted P, Iversen T Z, et al. Long-Lasting complete responses in patients with metastatic melanoma after adoptive cell therapy with tumor-infiltrating lymphocytes and an attenuated il2 regimen. Clin Cancer Res 2016; 22:3734-45. doi:10.1158/1078-0432.CCR-15-1879.

[2] Ekeruche-Makinde J, Clement M, Cole D K, Edwards E S J, Ladell K, Miles J J, et al. T-cell receptor-optimized peptide skewing of the T-cell repertoire can enhance antigen targeting. J Biol Chem 2012; 287:37269-81. doi:10.1074/jbc.M112.386409.

[3] Haney D, Quigley M F, Asher T E, Ambrozak D R, Gostick E, Price D A, et al. Isolation of viable antigen-specific CD8+ T cells based on membrane-bound tumor necrosis factor (TNF)-alpha expression. J Immunol Methods 2011; 369:33-41. doi: 10.1016/j.jim.2011.04.003.Isolation.

[4] Donia M, Kjeldsen J W, Andersen R, Westergaard M C W, Bianchi V, Legut M, et al. PD-1+ polyfunctional T cells dominate the periphery after tumor-infiltrating lymphocyte therapy for cancer. Clin Cancer Res 2017:clincanres.1692.2016. doi:10.1158/1078-0432.CCR-16-1692.

[5] Wooldridge L, Ekeruche-Makinde J, Van Den Berg H A, Skowera A, Miles J J, Tan M P, et al. A single autoimmune T cell receptor recognizes more than a million different peptides. J Biol Chem 2012; 287:1168-77.

[6] Szomolay B, Liu J, Brown P E, Miles J J, Clement M, Llewellyn-Lacey S, et al. Identification of human viral protein-derived ligands recognized by individual MHCI-restricted T-cell receptors. Immunol Cell Biol 2016; 94:573-82. doi:10.1038/icb.2016.12.

[7] Ekeruche-Makinde J, Miles J J, van den Berg H A, Skowera A, Cole D K, Dolton G, et al. Peptide length determines the outcome of TCR/peptide-MHCI engagement. Blood 2013; 121:1112-23. doi:10.1182/blood-2012-06-437202.

[8] Tungatt K, Bianchi V, Crowther M D, Powell W E, Schauenburg A J, Trimby A, et al. Antibody stabilization of peptide-MHC multimers reveals functional T cells bearing extremely low-affinity TCRs. J Immunol 2015; 194:463-74.

[9] Legut M, Dolton G, Mian A A, Ottmann O, Sewell A. CRISPR-mediated TCR replacement generates superior anticancer transgenic T-cells. Blood 2017:blood-2017-05-787598. doi:10.1182/blood-2017-05-787598.

[10] Hundemer M, Schmidt S, Condomines M, Lupu A, Hose D, Moos M, et al. Identification of a new HLA-A2-restricted T-cell epitope within HM1.24 as immunotherapy target for multiple myeloma. Exp Hematol 2006; 34:486-96. doi:10.1016/j.exphem.2006.01.008.

1-22. (canceled)

23. A composition comprising two or more peptides, wherein the amino acid sequence of each peptide consists of an amino acid sequence that is at least 80% identical to an amino acid sequence selected from the group consisting of SEQ ID NO: 80, SEQ ID NO: 77, SEQ ID NO: 76, SEQ ID NO: 78, SEQ ID NO: 79, SEQ ID NO: 81, SEQ ID NO: 82, SEQ ID NO: 83, SEQ ID NO: 84, and SEQ ID NO: 85.

24. The composition of claim 23, wherein the amino acid sequence of each peptide consists of an amino acid sequence that is at least 90% identical to an amino acid sequence selected from the group consisting of SEQ ID NO: 80, SEQ ID NO: 77, SEQ ID NO: 76, SEQ ID NO: 78, SEQ ID NO: 79, SEQ ID NO: 81, SEQ ID NO: 82, SEQ ID NO: 83, SEQ ID NO: 84, and SEQ ID NO: 85

25. The composition of claim 23, wherein the amino acid sequence of each peptide consists of an amino acid sequence selected from the group consisting of SEQ ID NO: 80, SEQ ID NO: 77, SEQ ID NO: 76, SEQ ID NO: 78, SEQ ID NO: 79, SEQ ID NO: 81, SEQ ID NO: 82, SEQ ID NO: 83, SEQ ID NO: 84, and SEQ ID NO: 85

26. The composition of claim 23, wherein the amino acid sequence of at least one peptide consists of the amino acid sequence of SEQ ID NO: 80 and/or the amino acid sequence of at least one peptide consists of the amino acid sequence of SEQ ID NO: 77.

27. The composition of claim 23 further comprising an adjuvant.

28. A pharmaceutical composition comprising the composition of claim 23 and a pharmaceutically acceptable carrier.

29. A method of treating cancer in a subject in need thereof, the method comprising administering the composition of claim 23 to the subject.

30. The method of claim 29, wherein the cancer is selected from the group consisting of: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, blood, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, and tonsil cancer.

31. A method of preparing a T-cell population comprising stimulating and amplifying T-cells ex vivo using the composition of claim 23.

32. A method of treating cancer in a subject in need thereof, the method comprising administering a T-cell population obtained from the method of claim 31 to the subject.

33. The method of claim 32, wherein the cancer is selected from the group consisting of: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, blood, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, and tonsil cancer.

34. A method of preparing an antigen presenting cell population, comprising introducing into an antigen presenting cell ex vivo a peptide as set forth in claim 23, or a nucleic acid sequence encoding the peptide.

35. An antigen presenting cell population obtained by the method of claim 34.

36. A method of preparing a T-cell population comprising stimulating and amplifying T-cells ex vivo using a peptide, wherein the amino acid sequence of the peptide consists of an amino acid sequence that is at least 80% identical to an amino acid sequence selected from the group consisting of SEQ ID NO: 80, SEQ ID NO: 77, SEQ ID NO: 76, SEQ ID NO: 78, SEQ ID NO: 79, SEQ ID NO: 81, SEQ ID NO: 82, SEQ ID NO: 83, SEQ ID NO: 84, and SEQ ID NO: 85.

37. The method of claim 36, wherein the amino acid sequence of the peptide consists of an amino acid sequence that is at least 90% identical to an amino acid sequence selected from the group consisting of SEQ ID NO: 80, SEQ ID NO: 77, SEQ ID NO: 76, SEQ ID NO: 78, SEQ ID NO: 79, SEQ ID NO: 81, SEQ ID NO: 82, SEQ ID NO: 83, SEQ ID NO: 84, and SEQ ID NO: 85

38. The method of claim 36, wherein the amino acid sequence of the peptide consists of an amino acid sequence selected from the group consisting of SEQ ID NO: 80, SEQ ID NO: 77, SEQ ID NO: 76, SEQ ID NO: 78, SEQ ID NO: 79, SEQ ID NO: 81, SEQ ID NO: 82, SEQ ID NO: 83, SEQ ID NO: 84, and SEQ ID NO: 85

39. A method of treating cancer in a subject in need thereof, the method comprising administering a T-cell population obtained by the method of claim 36 to the subject.

40. The method of claim 39, wherein the cancer is selected from the group consisting of: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, blood, tonsil, spleen, neuroma, von Hippel-Lindau disease, Zollinger-Ellison syndrome, adrenal cancer, anal cancer, bile duct cancer, bladder cancer, ureter cancer, glioma, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, muscle cancer, Paget's disease, cervical cancer, rectal cancer, esophagus cancer, gall bladder cancer, cholangioma cancer, head cancer, eye cancer, nasopharynx cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, myeloma, multiple myeloma, ovarian cancer, endocrine cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, and tonsil cancer.
